(19) United States
(12) Patent Application Publication
(10) Pub. No.: US 2022/0177890 A1
(43) Pub. Date: Jun. 9, 2022

(54) HYBRID NUCLEIC ACID SWITCHES

(71) Applicant: The United States of America,as represented by the Secretary,Department of Health and Human Services, Bethesda, MD (US)

(72) Inventors: Bruce A. Shapiro, Gaithersburg, MD (US); Paul J. Zakrevsky, Baltimore, MD (US)

(73) Assignee: The United States of America,as represented by the Secretary,Department of Health and Human Services, Bethesda, MD (US)

(21) Appl. No.: 17/602,204

(22) PCT Filed: Apr. 10, 2020
(86) PCT No.: PCT/US2020/027637

§ 371 Date: Oct. 7, 2021

Related U.S. Application Data
(60) Provisional application No. 62/832,171, filed on Apr. 10, 2019.

(51) Int. Cl. C12N 15/113; A61K 9/51

(52) U.S. Cl. CPC C12N 15/1135; C12N 2310/113; C12N 15/1136; A61K 9/51

(57) ABSTRACT

Disclosed are DNA/RNA hybrid nucleic acid nanoparticles comprising at least one trigger toehold or at least one exchange toehold, wherein each at least one trigger toehold and the at least one exchange toehold independently comprise DNA and/or RNA, and at least one single stranded RNA output strand, wherein no portion of the at least one trigger toehold hybridizes to any portion of the at least one output strand, the at least one trigger toehold is complementary and hybridizes to a first target sequence when the nanoparticle is in the presence of the first target sequence, and the nanoparticle does not contain the target sequence. Related pharmaceutical compositions, methods of treating a patient with a disease or condition, and methods of diagnosing a patient with a disease or condition are also disclosed.

### CROSS-REFERENCE TO RELATED APPLICATIONS

This application claims priority to U.S. Provisional Patent Application No. 62/832,171, filed Apr. 10, 2019, which is incorporate herein by reference.

### FEDERALLY SPONSORED RESEARCH OR DEVELOPMENT

This invention was made with Government support under project number Z01BC01106111 by the National Institutes of Health, National Cancer Institute. The Government has certain rights in the invention.

### INCORPORATION-BY-REFERENCE OF MATERIAL SUBMITTED ELECTRONICALLY

Incorporated by reference in its entirety herein is a computer-readable nucleotide/amino acid sequence listing submitted concurrently herewith and identified as follows: One 12,512 Byte ASCII (Text) file named “748803_ST25.TXT,” dated Apr. 8, 2020.

### BACKGROUND OF THE INVENTION

RNA nanoparticles may be useful for a variety of nanobiological applications. Such applications may include, for example, the delivery of functional moieties, such as ligand binding motifs or gene expression regulators. Despite advancements in the field of RNA nanoparticles, a variety of challenges to the successful application of RNA nanoparticles remain. For example, distinguishing aberrant cells in need of therapeutic treatment and limiting the activity of deliverable nucleic acid constructs to these specific cells remains a challenge. Accordingly, there exists an unmet need for improved RNA nanoparticles, including designed and characterized nanoparticles able to generate and/or release sequence-specific oligonucleotide constructs in a conditional manner based on the presence or absence of RNA trigger molecules.

### BRIEF SUMMARY OF THE INVENTION

An embodiment of the invention provides DNA/RNA hybrid nucleic acid nanoparticles comprising at least one trigger toehold or at least one exchange toehold, wherein each at least one trigger toehold and the at least one exchange toehold independently comprise DNA and/or RNA, and at least one single stranded RNA output strand, wherein no portion of the at least one trigger toehold hybridizes to any portion of the at least one output strand, the at least one trigger toehold is complementary and hybridizes to a first target sequence when the nanoparticle is in the presence of the first target sequence, and the nanoparticle does not contain the target sequence.

Another embodiment of the invention provides compositions comprising the inventive nanoparticles.

Further embodiments of the invention provide methods of treating a patient with a disease or condition comprising administering the inventive nanoparticles or compositions to the patient.

Still another embodiment of the invention provides methods of diagnosing a patient with a disease or condition comprising administering the inventive nanoparticles or compositions to the patient.

### BRIEF DESCRIPTION OF THE SEVERAL VIEWS OF THE DRAWINGS

FIG. 1 is a schematic showing nanoparticle switches according to embodiments of the invention. On the left, the “beacon-derived switch” is a bimolecular system able to release a single-stranded output oligo when a particular trigger sequence was recognized by its internal diagnostic toehold. The “adjacent-targeting” (middle) and “inducible activation” (right) RNA/DNA hybrid switches are systems that require a cognate pair of constructs to generate a double-stranded RNA output upon recognition of a trigger molecule.

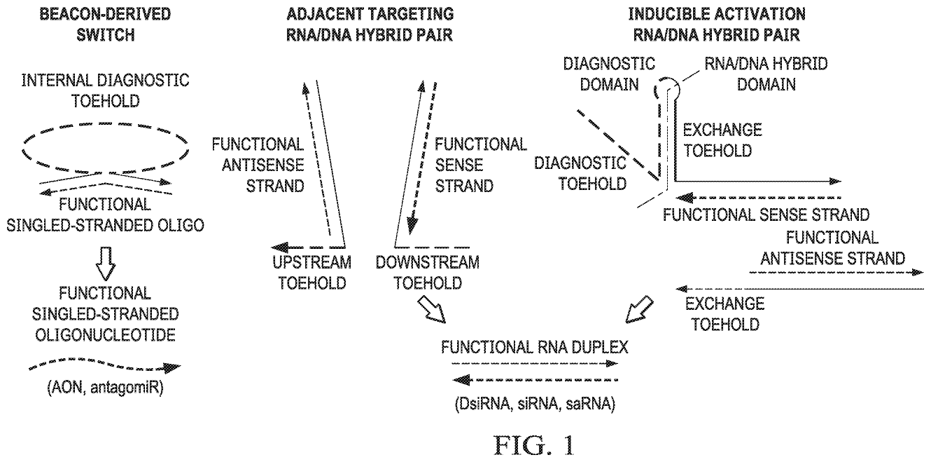

FIG. 2A is a schematic showing “traditional” molecular beacon with fluorescence-based unimolecular diagnostic systems that adopt an initial loop structure. Hybridization of a trigger sequence complementary to the hairpin loop opens the hairpin and alters the fluorescence of the beacon by separating a fluorophore/quencher pair.

FIG. 2B is a schematic showing a “beacon-derived” biomolecular switch system according to an embodiment of the invention composed of a diagnostic strand and an output strand. The output strand is hybridized to the 5′ and 3′ ends of the diagnostic strand creating a large bulge in the diagnostic strand. This bulge acts as an internal toehold. Hybridization of a trigger to this toehold region forms a persistent helix that outcompetes the internal pairing between the diagnostic and output strands, causing release of the output strand.

FIG. 2C is an image of a 10% acrylamide non-denaturing polyacrylamide gel electrophoreses (PAGE) after staining with ethidium bromide showing the result of analysis of the conditional function of the beacon-derived switch. The beacon switch was assembled from the diagnostic and output strands. Addition of the trigger RNA to the pre-assembled beacon switch releases an output strand (box) and shows generation of the expected waste byproduct. The fraction of output strand released was estimated by comparing the density of the output band to the output strand control lane of the same initial concentration. All samples were incubated for 30 minutes at 37° C.

FIG. 3A is a schematic showing how “traditional” switches function wherein RNA/DNA hybrid pairs hybridize between the single stranded toeholds of a sense hybrid (ski) and antisense hybrid (all) which causes a thermodynamically driven strand exchange that generates a dsRNA duplex and DNA waste byproduct.

FIG. 3B shows the “adjacent targeting” RNA/DNA hybrid system of an embodiment of the invention functions by requiring a hybrid pair as well as a specific RNA trigger sequence. The hybrid pair's respective toeholds bind to regions of the trigger that are upstream and downstream from one another (i.e., adjacent or just with a few nucleotides between the binding sites). Anchoring the cognate hybrids in close proximity leads to initiation of the thermodynamically favorable strand exchange reaction and dsRNA release.

FIG. 4A is a schematic showing how the inducible hybrid system according to an embodiment of the invention functions. The sense hybrid sH_{\hat{ }CTGF.12/8} contains a responsive DNA hairpin composed of a 12 base pair stem and an 8 nucleotide loop, and is flanked by an extended 5′ single strand that acts as a trigger toehold. Trigger hybridization to the trigger toehold progresses through the hairpin stem and unzips the hairpin. This action liberates a previously sequestered exchange toehold within sH_{\hat{ }CTGF.12/8} which can then hybridize with the complementary exchange toehold of the cognate antisense hybrid, aH_{\hat{ }CTGF-cgnt.12}. Hybridization of these exchange toeholds initiates strand exchange and releases a double stranded (ds)RNA output/product.

FIG. 4B is an image of an 8% acrylamide non-denaturing PAGE after staining with ethidium bromide showing the result of the analysis of the switch system shown in FIG. 4A. DsiRNA release is observed when the sense and antisense hybrids are co-incubated in the presence of trigger (box). Formation of the expected waste product is observed by comparison to a control assembly of the s′ and a′ DNA strands with the trigger molecule. All samples were incubated for 30 minutes at 37° C.

FIG. 4C shows the results of Förster resonance energy transfer (FRET) analysis that was performed as another method to verify conditional dsRNA formation. sH_{\hat{ }CTGF.12/8} was assembled using a 3′-6-FAM (ex/em 495/520 nm) labeled sense RNA strand. aH_{\hat{ }CTGF-cgnt.12} was assembled using a 5′-ALEXAFLUOR546 (ex/em 555/570 nm) labeled antisense RNA strand. The hybrids were mixed and incubated at 37° C. for one hour in the presence or absence of the RNA trigger. Fluorescence emission spectra were recorded at t=0 and t=60 minutes using excitation at 475 nm.

FIG. 5A is a schematic showing four different sense hybrids according to an embodiment of the invention that are responsive to the CTGF trigger. The hairpins of each hybrid differed in the size of their loop or the length of their stem. Two different cognate antisense hybrids were designed and differ in the length of their single-stranded toehold. Sequence regions are indicated by lowercase letters to convey sequence identity or sequence complementarity.

FIG. 5B is an image of a 10% acrylamide non-denaturing PAGE after staining with ethidium bromide showing the result of the analysis of DsiRNA release in the presence and absence of trigger for each sense hybrid paired with a cognate antisense hybrid exhibiting a 12 nucleotide toehold (aH_{\hat{ }CTGF-cgnt.12}). Each sense hybrid and the DsiRNA control contained a 3′-6-FAM labeled sense RNA strand for visualization and quantification. Gels depict samples that were incubated for 30 minutes at 37° C.

FIG. 5B is an image of a 10% acrylamide non-denaturing PAGE after staining with ethidium bromide showing the result of the analysis of DsiRNA release in the presence and absence of trigger for each sense hybrid paired with a cognate antisense hybrid exhibiting a 16 nucleotide toehold (aH_{\hat{ }CTGF-cgnt.16}). Each sense hybrid and the DsiRNA control contained a 3′-6-FAM labeled sense RNA strand for visualization and quantification. Gels depict samples that were incubated for 30 minutes at 37° C.

FIG. 6A is a schematic showing a trigger-repressive hybrid system according to an embodiment of the invention that was designed. The antisense hybrid, aH_{vKRAS}, was designed to repress strand exchange in the presence of a KRAS trigger sequence. If the trigger is absent, the toehold of aH_{vKRAS} is freely accessible and can promote dsRNA release. If the trigger is present, its hybridization to the diagnostic toehold of aH_{vKRAS} results in a structural rearrangement that blocks access to the exchange toehold and prevents interaction with the cognate sense hybrid, sH_{vKRAS-cgnt}.

FIG. 6B is an image of a 10% acrylamide non-denaturing PAGE after staining with ethidium bromide showing the result of the analysis of the conditional function of the switch system shown in FIG. 6A. DsiRNA release from the aH_{vKRAS}/sH_{vKRAS-cgnt} pair was examined in three contexts: in the absence of the KRAS trigger (middle lane), when sH_{vKRAS-cgnt} and the KRAS trigger are premixed and added simultaneously to aH_{vKRAS} (2nd lane from right), or when aH_{vKRAS} and the KRAS trigger are preincubated for 5 minutes prior to sH_{vKRAS-cgnt} addition (right lane). The KRAS trigger was added in 3-fold excess in both cases. The depicted gel shows samples incubated for 180 minutes at 37° C. once all components were present.

FIG. 6C is a schematic showing a multi-trigger system according to an embodiment of the invention that was designed in which each RNA/DNA hybrid contains a responsive DNA structural element. sH_{\hat{ }CTGF.20/8} (activated by CTGF) was paired with aH_{vKRAS} (repressed by KRAS). Co-incubation of the two hybrids results in no interaction. Both hybrids and the CTGF trigger are required for dsRNA release, while the presence of the KRAS trigger will inhibit strand exchange.

FIG. 6D is an image of a 10% acrylamide non-denaturing PAGE after staining with ethidium bromide showing the result of the analysis of the multi-trigger system shown in FIG. 6C. The fraction of DsiRNA released is indicated in the gel depicted, in the presence of indicated trigger combinations following 30 minute incubation at 37° C. The sH and aH hybrids were present at equimolar concentration, while the triggers were added at a 2-fold or 3-fold excess, as indicated. In samples when both triggers are present, they were added to premixed hybrids sequentially (KRAS followed by CTGF).

FIG. 7 shows free energy calculations of the predicted initial and final states for the beacon-derived switch interacting with the KRAS trigger. The final state shows a structure in which only the 5′ end of the output strand was separated from the diagnostic strand. Energy calculations and secondary structure predictions were performed using HyperFold (see Bindewald et al., Nano Lett., 16: 1726-1735 (2016)).

FIG. 8 shows free energy calculations of the initial and final states for the “+0 bp” adjacent targeting hybrid system. Energy calculations and secondary structure predictions were performed using Hyperfold.

FIG. 9 is an image of a 12% acrylamide non-denaturing PAGE after staining with ethidium bromide showing the result of the analysis of cognate pairs of adjacent targeting hybrids for their ability to release Dicer substrate iRNA (DsiRNA) product as described in FIG. 3A. Each sense hybrid and the DsiRNA control assembly contained a 3′-6-carboxyfluorescein succinimidyl (FAM) donor fluorophore sense RNA strand for visualization. The pairs of constructs differ in the number of DNA nucleotides inserted between the single-strand trigger toeholds and the RNA/DNA hybrid duplex. These inserted nucleotides are complementary between cognate hybrids, resulting in either 0, +1, +2, +3 or +4 DNA base pairs that can seed the strand exchange. The presence or absence of each component was indicated above each lane. The samples in the gel depicted were all incubated for 180 minutes at 37° C.

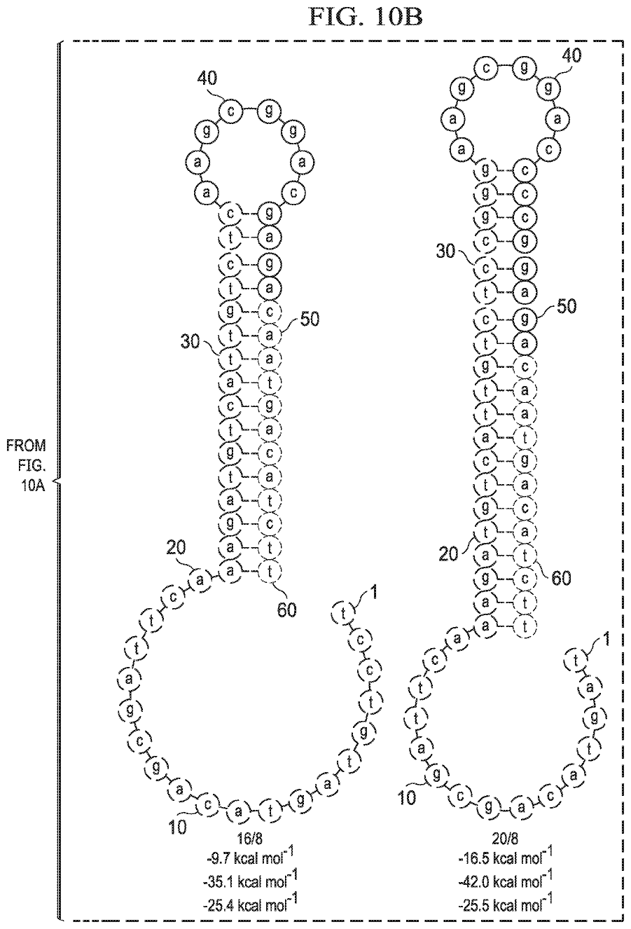

FIG. 10 shows the free energy calculations of the responsive DNA hairpin elements of variant sH_{\hat{ }CTGF} hybrids as predicted by Hyperfold. Free energies are given for the initial hairpin/toehold structure (structures shown, ΔGₕₐᵢᵣₚᵢₙ), the hairpin/toehold bound to the CTGF trigger (ΔG_{hairpin+trigger}), as well as the difference in free energy between these two states (ΔΔG). Nucleotides that define the hybrids' exchange toehold are labeled in each hairpin loop. The nucleotides that are complementary to the trigger are in grey without the black outline. The nucleotides between the region complementary to the trigger and the exchange toehold have a black outline. The distance between exchange toehold and nucleotides complementary to the trigger increases within the structures moving from left to right.

FIG. 11 shows alternate structures adopted by the responsive hairpin/toehold region of aH_{vKRAS} as predicted by Hyperfold. Stretches of poly-A were inserted into the loop sequence of each state to avoid pseudoknots and examine the energies of the two distinct hairpins. The “on” state is initially energetically preferred in absence of trigger, but the “off” state structure was stabilized by hybridization of the KRAS trigger.

FIG. 12 are images of an acrylamide non-denaturing PAGE after staining with ethidium bromide showing the result of the analysis of the extent of dsRNA release of trigger-responsive nanoparticles in the presence of various trigger sequences. The aH_{vKRAS}/sH_{vKRAS.cgnt} pair (left) was designed to repress dsRNA release in presence of the KRAS trigger. The sH_{\hat{ }CTGF.20/8}/aH_{\hat{ }CTGF.cgnt12} pair (right) is designed to induce dsRNA release in presence of the CTGF trigger. aH*_{vKRAS} and its corresponding DsiRNA control contain a 5′-ALEXAFLUOR546 labeled RNA antisense strand for visualization, while SH*_{\hat{ }CTGF.20/8} and the corresponding DsiRNA control contain 3′-6-FAM labeled sense RNA strands.

FIG. 13 is a schematic (top) of a repressive hybrid system according to an embodiment of the invention and an image of an acrylamide non-denaturing PAGE (bottom) after staining with ethidium bromide showing the result of the analysis of the nanoparticles shown in top schematic. Not only did the CTGF-repression system respond to a different trigger sequence than the vKRAS system, but the vCTGF system was designed as the mirror opposite of the KRAS-repression system. The vCTGF system contained the responsive DNA structural element on the 5′ end of the sense hybrid, whereas the responsive element of the vKRAS system was on the 3′ end of the antisense hybrid. The sense hybrid and DsiRNA control contained a 3′-6-FAM labeled sense RNA for visualization.

FIG. 14A is a schematic showing the method according to an embodiment of the invention. The aH_{vKRAS} and sH_{\hat{ }CTGF.20/8} hybrids were initially premixed at equimolar concentrations. Multiple tubes of the cognate KRAS and CTGF triggers were also premixed, at various relative concentrations, ranging from 0×-3× the concentration of the hybrid concentration. An aliquot of the hybrid mixture was then added to each tube containing triggers and incubated at 37° C. for 30 minutes. The experiment was designed to reduce any kinetic bias on the system based on the order of construct addition to the reaction.

FIG. 14B is an image of an acrylamide non-denaturing PAGE after staining with ethidium bromide showing the result of the analysis of the extent of DsiRNA release for each trigger concentration. aH*_{vKRAS} and the DsiRNA control were assembled with a 5′-ALEXAFLUOR-546 labeled RNA antisense strand for visualization and quantitation.

FIG. 15 is a schematic (top) of a 3-piece trigger-inducible RNA/DNA system according to an embodiment of the invention and an image of an acrylamide non-denaturing PAGE (bottom) after staining with ethidium bromide showing the result of the analysis of the ability of the nanoparticles shown in top schematic to release dsRNA in a conditional fashion. The sense hybrids and DsiRNA control contain a 3′-6-FAM labeled sense RNA for visualization.

FIG. 16 is set of a schematics (top) of a 3-piece trigger-repressible RNA/DNA hybrids according to embodiments of the invention and an image of an acrylamide non-denaturing PAGE (bottom) after staining with ethidium bromide showing the result of the analysis of the nanoparticles shown in top schematic. The nanoparticles were examined for their ability to release dsRNA in a conditional fashion. The different 3-piece aH_{vKRAS} hybrids were created by inserting a nick in the stem of the responsive DNA element. Each 3-piece aH_{vKRAS} hybrid was partnered with sH_{vKRAS.cgnt}. The ability of the 3-piece hybrids to maintain conditional function decreases as the nick was moved further away from the apical loop of the DNA hairpin.

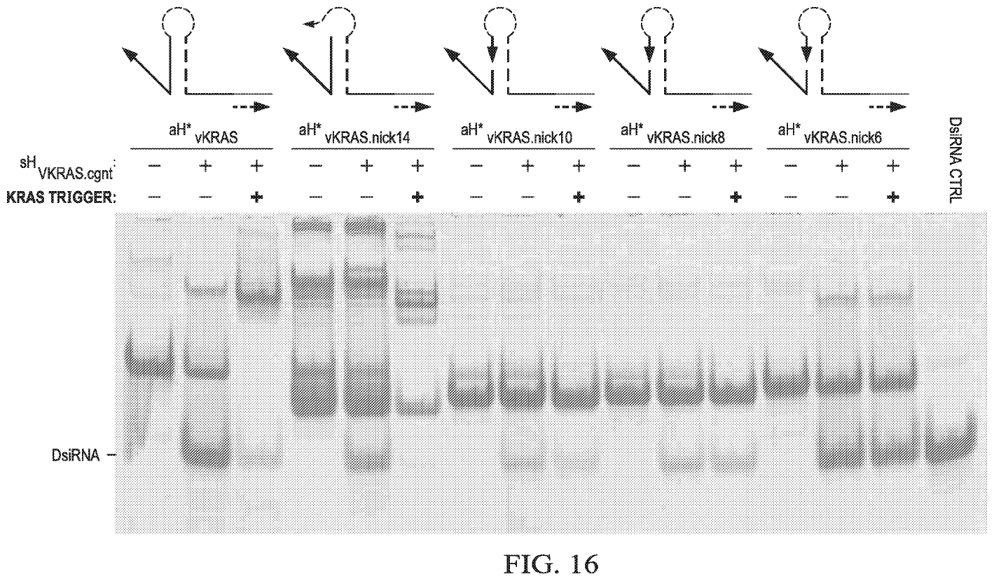

FIG. 17 is set of schematics (top) of hybrids according to embodiments of the invention and a set of graphs showing the results of FRET analysis (bottom). FRET time course experiments were used to monitor dsRNA release for a hybrid system where the sense hybrid requires CTGF to become active, while the function of the antisense hybrid can be repressed by interaction with KRAS. Hybrids sH_{\hat{ }CTGF.20/8} and aH_{vKRAS}. were combined to a final concentration of 500 nM final (left) in the absence of any trigger molecules, (middle) in the presence of a the 2-fold excess of CTGF, or (right) in a context when a 3-fold excess of KRAS followed by a 2-fold excess of CTGF are added to the hybrids in a sequential fashion.

FIG. 18 shows two sets of schematics of the inducible hybrid system according to embodiments as disclosed herein and a gel picture: (top portion of figure) in the absence of trigger, no strand exchange occurs; (bottom portion of figure) in the presence of trigger, strand exchange occurs releasing product to form dsRNA; and (bottom right of figure) a gel with lanes labeled at the top, showing absence of dsRNA in the absence of trigger and dsRNA formation in the presence of trigger, fragment sizes are compared to positive control dsRNA in the far right lane. Gel results show 500 nM concentration of hybrids, with 2-fold excess of trigger molecule (1 uM) in buffer.

FIG. 19 shows the results of experiments performed in 5 μg of extracted total cellular RNA (purified by column based RNA extraction kit). Each graph shows increase in fluorescent product released by the inducible hybrid system according to embodiments as disclosed herein when trigger is present, based on accompanying gel results (appearing to the right of the graphs) as detected and measured by fluorescence.

FIG. 20 shows the results of experiments performed in cell lysate. The graphs show increase in fluorescent product released by the inducible hybrid system according to embodiments as disclosed herein when trigger is present. FIG. 20 further shows that fluorescent product is released in cell lysate, as well as buffer, with the accompanying gel results provided in histogram format as detected and measured by fluorescence.

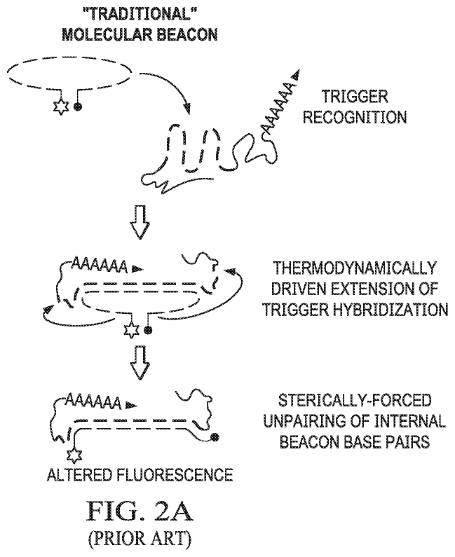

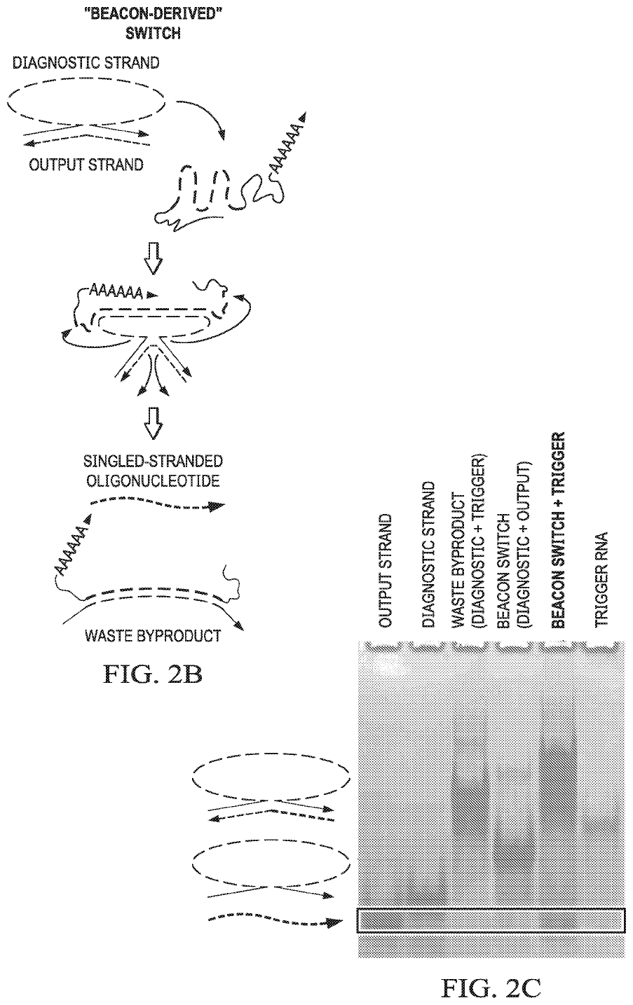

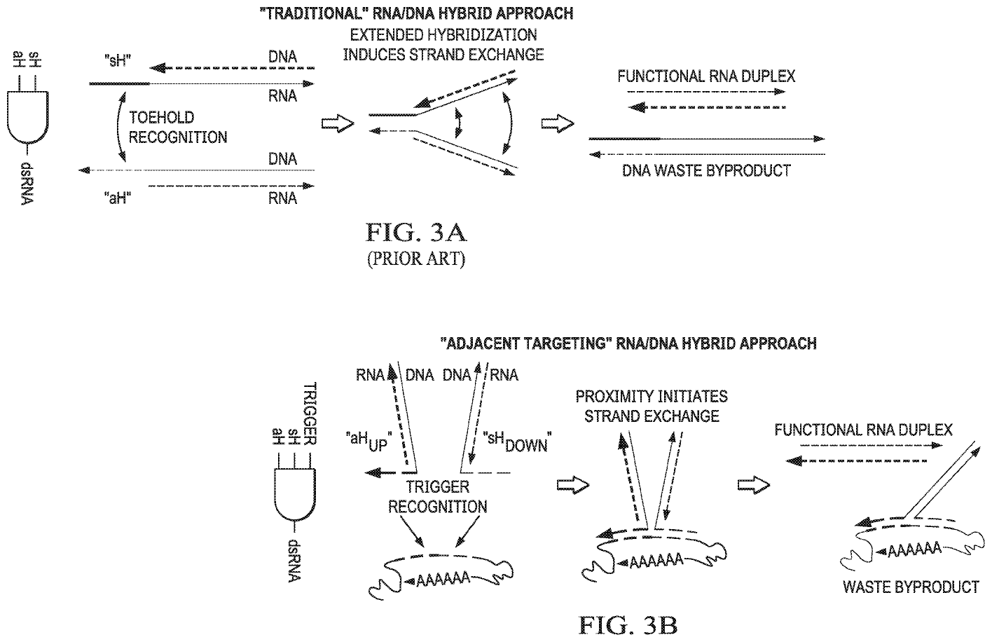

The symbol “A” as it appears in the nucleic acid schematics of the Figures, including but not limited to, FIGS. 2A, 2B, 3B, 4A, 5A, 6A, 6C, 14A and 18 represents an arbitrary base.

### DETAILED DESCRIPTION OF THE INVENTION

The inventive nanoparticles provide any one or more of a variety of advantages. For example, one advantage of the nanoparticles is that the “diagnostic region” or the sequence that binds the target or trigger molecule (e.g., mRNA or fragment thereof) is structurally separated from the payload or output strand(s). This separation allows for input and output sequences to be completely decoupled and imparts no sequence constraints on one another. This allows for changes in the diagnostic region of the nanoparticles to be independent of the sequence of the payload region of the nanoparticles.

Another advantage of the inventive nanoparticles is that the RNA strands do not require additional 2′-modifications for protection from ribonucleases. This protection is not required because the RNA strands are initially bound within RNA/DNA hybrid duplexes to provide resistance from ribonuclease degradation. Adding 2′-modifications are not desirable because these modifications can increase the costs and reduce efficiency of commercial oligonucleotide synthesis.

Yet another advantage of the inventive nanoparticles is that the nanoparticles allow for a degree of conditional control typically only observed in systems designed for conditional generation of sequence specific dsRNA by demonstrating that conditional dsRNA release can not only be induced but also repressed upon interaction with an RNA trigger (also referred to herein as the first or second target sequences) culminating in a cognate pair of RNA/DNA hybrid constructs for which dsRNA release is under the control of multiple input triggers/targets.

Additionally, the inventive nanoparticles provide flexibility to the user because they can deliver several payloads/output strands (e.g., single stranded or double stranded oligonucleotides) under various conditions (e.g., diagnostic or treatment methods, biomarker mediated induction or repression).

In an embodiment the invention provides a DNA/RNA hybrid nucleic acid nanoparticle comprising: (a) at least one trigger toehold or at least one exchange toehold, wherein each at least one trigger toehold and the at least one exchange toehold independently comprise DNA and/or RNA; and (b) at least one single stranded RNA output strand, wherein no portion of the at least one trigger toehold hybridizes to any portion of the at least one output strand, the at least one trigger toehold is complementary and hybridizes to a first target sequence when the nanoparticle is in the presence of the first target sequence, and the nanoparticle does not contain the target sequence.

In an embodiment, at least one output strand separates from the nanoparticle when the at least one trigger toehold hybridizes to the first target sequence. The binding of the at least one trigger toehold to the first target sequence does not necessarily cause the release of the output strand/payload but the binding of the at least one trigger toehold to the first target sequence may allow for the nanoparticles to configure in a way such that the payload can be released.

In an embodiment, the nanoparticles do not include additional 2′ modified nucleotides. In preferred embodiment, the at least one output strand does not comprise 2′ modified nucleotides. As discussed above, modifying 2′ nucleotides reduces ribonuclease degradation. Because the nanoparticles are DNA/RNA hybrids, the 2′ modifications are not needed.

In an embodiment, the at least one trigger toehold forms a loop that does not contain the at least one output strand and the nanoparticle comprises at least one strand that is complementary to the at least one output strand.

In a further embodiment, the nanoparticle comprises a sense construct and an antisense construct. In an embodiment, the sense construct and the antisense construct are not connected to each other and are two separate constructs.

In another embodiment, the sense construct comprises a first trigger toehold and a first output strand, the antisense construct comprises a second trigger toehold and a second output strand, and the first trigger toehold and the second trigger toehold are complementary to adjacent positions within the first target sequence.

In an embodiment, first trigger toehold and the second trigger toehold hybridize to adjacent positions within the first target sequence, the first output strand hybridizes to the second output strand and forms a double stranded output strand that separates from the nanoparticle.

In an embodiment, the sense construct comprises a first DNA strand comprising a sequence that is complementary to the first output strand and the antisense construct comprises a second DNA strand comprising a sequence that is complementary to the second output strand, wherein the first DNA strand is connected to the first trigger toehold and the second DNA strand is connected to the second trigger toehold.

In an embodiment, the first DNA strand of the sense construct comprises from about 1 to about 100 nucleic bases, or any number between 1 and 100 (i.e., from about 1 to about 90, from about 1 to about 80, from about 1 to about 70, from about 1 to about 60, from about 1 to about 50, from about 1 to about 40, from about 1 to about 30, from about 1 to about 20, from about 1 to about 10, or 9, 8, 7, 6, 5, 4, 3, 2 or 1), between the first trigger toehold and the sequence that is complementary to the first output strand.

In another embodiment, the second DNA strand of the antisense construct comprises from about 1 to about 100 nucleic bases, or number between 1 and 100 (i.e., from about 1 to about 90, from about 1 to about 80, from about 1 to about 70, from about 1 to about 60, from about 1 to about 50, from about 1 to about 40, from about 1 to about 30, from about 1 to about 20, from about 1 to about 10, or 9, 8, 7, 6, 5, 4, 3, 2 or 1), between the second trigger toehold and the sequence that is complementary to the second output strand.

In an embodiment, the sense construct comprises at least one hairpin loop comprising a helical stem and a loop. In another embodiment, the antisense construct comprises at least one hairpin loop comprising a helical stem and a loop. The helical stems and loops can be any size. If the exchange toeholds are within the hairpin loops of the sense or antisense constructs, then the helical stem and/or loop must be large enough to sequester the exchange toeholds (i.e., must be at least big enough for the entire toeholds to be within the hairpin loop structure). In an embodiment, the helical stem of the hairpin loop comprises from about 6 to about 50, or any number between (i.e., from about 12 to about 50, from about 12 to about 20, from about 12 to about 16), base pairs. In an embodiment, loop of the hairpin loop comprises from about 3 to about 30, or any number between (i.e., from about 5 to about 25, from about 8 to about 20, from about 12 to about 20), nucleotides.

In another embodiment, the sense construct comprises a first trigger toehold, a first exchange toehold, a first output strand, and a first DNA strand comprising a sequence that is complementary to the first output strand.

In another embodiment, the antisense construct comprises a second output strand and a second exchange toehold that is connected to a second DNA strand comprising a sequence that is complementary to the second output strand.

In an embodiment, an exchange toehold is within a helical stem of the hairpin loop of the sense construct. The first exchange toehold can be within the helical stem of the hairpin loop of the sense construct. In an embodiment, an exchange toehold is within a helical stem of the hairpin loop of the antisense construct. The second exchange toehold can be within the helical stem of the hairpin loop of the antisense construct.

In an embodiment, an exchange toehold (e.g., first or second exchange toehold) is not within a helical stem of the hairpin loop of the sense construct. In an embodiment, an exchange toehold (e.g., first or second exchange toehold) is not within a helical stem of the hairpin loop of the antisense construct.

In another embodiment, when the at least one trigger toehold hybridizes to the first target sequence, the hairpin loop is disrupted exposing the first exchange toehold such that the first exchange toehold can bind to the second exchange toehold allowing the first output strand to hybridize to the second output strand and thereby release the double stranded RNA output strand.

In an embodiment, the first target sequence is not in proximity to the sense construct, the hairpin loop is not disrupted and the first exchange toehold is kept within the helical stem of the hairpin loop, and a double stranded RNA output strand is not created by the first output strand hybridizing to the second output strand and a double stranded RNA output strand is not released by the nanoparticle.

In an embodiment, when the at least one trigger toehold hybridizes to the first target sequence, the hairpin loop is disrupted sequestering the first exchange toehold, a double stranded RNA output strand is not created by the first output strand hybridizing to the second output strand and a double stranded RNA output strand is not released by the nanoparticle.

In an embodiment, the sense construct further comprises a first helical loop with a first helical stem and a first hairpin loop and the first exchange toehold is sequestered within the first helical stem, and the antisense construct further comprises a second helical loop with a second helical stem and a second hairpin loop and the second exchange toehold is not within the second helical loop. In this embodiment, the first exchange toehold is no longer sequestered within the first helical stem when the sense construct is hybridized to the first target sequence allowing it to bind to a complementary sequence. The second toehold becomes sequestered within a second helical loop when the antisense construct hybridizes to a second target sequence and therefore the second toehold cannot bind to a complementary sequence that is outside of the helical loop. In this situation, the ratio of the amount of the first target sequence to the amount of the second target sequence in proximity to the sense construct and antisense construct that is sufficient to result in hybridization of the first trigger toehold to the first target sequence or the second trigger toehold to the second target sequence impacts the binding kinetics between the first exchange toehold and the first target sequence and the second toehold and the second target sequence.

As used herein, “proximity” means that the target sequence is within the environment of the sense and antisense constructs such that the target sequence could bind to an exchange toehold on the sense or antisense construct.

The ratio of the first target sequence to the second target sequence can be any ratio. In an embodiment, the amount of the first target sequence to the amount of the second target sequence is from about 1:900 to about 900:1, from about 1:800 to about 800:1, from about 1:700 to about 700:1, from about 1:600 to about 600:1, from about 1:500 to about 500:1, from about 1:400 to about 400:1, from about 1:300 to about 300:1, from about 1:200 to about 200:1, from about 1:100 to about 100:1, from about 1:75 to about 75:1, from about 1:50 to about 50:1, from about 25:1 to about 1:25, from about 20:1 to about 1:20, from about 15:1 to about 1:15, from about 10:1 to about 1:10, from about 5:1 to about 1:5, from about 1:3 to about 3:1, or is about 1:1, about 1:3, or about 3:1.

The first target sequence can be a sequence that is naturally occurring. For example, the first target sequence is part of a RNA sequence. The RNA can be messenger (mRNA), ribosomal RNA (rRNA), or transfer RNA (tRNA). In an embodiment, the RNA sequence is a mRNA sequence.

The first target sequence can also be biomarker. Suitable biomarkers include CEA, HER2, bladder tumor antigen, thyroglobulin, alpha-fetaprotein, PSA, CA 125, CA19.9, CA15.3, leptin, prolactin, osteopontin, IGF-II, troponin, and b-type natriuretic peptide.

In an embodiment, the first target sequence is KRAS (SEQ ID NO:61), or a fragment thereof. In another embodiment, the first target sequence is CTGF (SEQ ID NO:59), or a fragment thereof.

The second target sequence can be a sequence that is naturally occurring. For example, the second target sequence is part of a RNA sequence. The RNA can be messenger (mRNA), ribosomal RNA (rRNA), or transfer RNA (tRNA). In an embodiment, the RNA sequence is a mRNA sequence.

The second target sequence can also be biomarker. Suitable biomarkers include CEA, HER2, bladder tumor antigen, thyroglobulin, alpha-fetaprotein, PSA, CA 125, CA19.9, CA15.3, leptin, prolactin, osteopontin, and IGF-II, troponin, and b-type natriuretic peptide.

In an embodiment, the second target sequence is KRAS (SEQ ID NO:61), or a fragment thereof. In another embodiment, the second target sequence is CTGF (SEQ ID NO:59), or fragment thereof.

An embodiment of the invention provides a set of DNA/RNA constructs that have sections that are complementary to each other and contain a payload comprised of a first and second DNA/RNA construct. The first DNA/RNA construct, sense hybrid, comprises a first toehold trigger region that is partially single-stranded in which it recognizes and binds to a target sequence (e.g., KRAS, CTGF) that is connected to sequence strand that forms a hairpin loop, which is connected to a second trigger region that is complementary to a portion of the first trigger region strand and complementary to the toehold region of the 2nd DNA/RNA construct, which then is connected to a double-stranded DNA/RNA hybrid duplex that contains half of the payload and is complementary to the DNA/RNA hybrid duplex on the 2^{nd} DNA/RNA construct. The second DNA/RNA construct, anti-sense hybrid, comprises a single-stranded toehold complementary to the 2^{nd} trigger region on the first DNA/RNA construct and a double-stranded region that is complementary to a portion of the first DNA/RNA construct, wherein the complementary 2^{nd} trigger region strand hybridizes to the toehold region of the 2nd DNA/RNA construct initiating the hybridization of the double stranded DNA/RNA hybrid regions of the 2 constructs which exchange and release a dsRNA payload (output strand) in the presence of the target sequence.

RNA interference (RNAi) substrate may include double-stranded RNA, siRNA, shRNA, or antisense RNA, or a portion thereof, or a mimetic thereof, that when administered to a mammalian cell results in a decrease (e.g., by about 10%, about 25%, about 50%, about 75%, or even about 90 to about 100%) in the expression of a target gene. Typically, an RNAi substrate comprises at least a portion of a target nucleic acid molecule, or an ortholog thereof, or comprises at least a portion of the complementary strand of a target nucleic acid molecule. In an embodiment, the RNAi substrate may comprise a small interfering RNA (siRNA), a short hairpin miRNA (shMIR), a microRNA (miRNA), Dicer substrate RNA (DsiRNA), or an antisense nucleic acid. In an embodiment, the siRNA may comprise, e.g., trans-acting siRNAs (tasiRNAs) and/or repeat-associated siRNAs (rasiRNAs). In another embodiment, the miRNA may comprise, e.g., a short hairpin miRNA (shMIR). In a preferred embodiment, the RNAi substrate comprises DsiRNA.

In an embodiment, the invention provides a method of treating a patient with a disease or condition, the method comprising administering any one of the inventive nanoparticles disclosed herein or the inventive compositions disclosed herein to the patient. If a first and second constructs are administered to the patient, they can be administered sequentially or concurrently.

In an embodiment, the invention provides a method of diagnosing a patient with a disease or condition, the method comprising (a) administering any one of the inventive nanoparticles disclosed herein or inventive compositions disclosed herein to the patient, and (b) observing the level of separated output strands in a patient sample and comparing the level of separated output strands to a threshold. The threshold level can be determined by one of skill in the art.

The inventive RNA nanoparticles can be formulated into a composition, such as a pharmaceutical composition. In this regard, the invention provides a pharmaceutical composition comprising any of the RNA nanoparticles described herein and a pharmaceutically acceptable carrier. The inventive pharmaceutical compositions containing any of the inventive RNA nanoparticles can comprise more than one inventive RNA nanoparticle, e.g., RNA nanoparticles comprising different functional moieties. Alternatively, the pharmaceutical composition can comprise an inventive RNA nanoparticles in combination with another pharmaceutically active agent(s) or drug(s), such as a chemotherapeutic agents, e.g., asparaginase, busulfan, carboplatin, cisplatin, daunorubicin, doxorubicin, fluorouracil, gemcitabine, hydroxyurea, methotrexate, paclitaxel, rituximab, vinblastine, vincristine, etc.

Preferably, the carrier is a pharmaceutically acceptable carrier. With respect to pharmaceutical compositions, the carrier can be any of those conventionally used for RNA nanoparticles. Methods for preparing administrable compositions are known or apparent to those skilled in the art and are described in more detail in, for example, Remington: The Science and Practice of Pharmacy, 22^{nd} Ed., Pharmaceutical Press (2012). It is preferred that the pharmaceutically acceptable carrier be one which has no detrimental side effects or toxicity under the conditions of use.

The choice of carrier will be determined in part by the particular inventive nanoparticle, the particular functional moiety (or moieties) attached to the nanoparticle, as well as by the particular method used to administer the inventive nanoparticle. Accordingly, there are a variety of suitable formulations of the pharmaceutical composition of the invention. Suitable formulations may include any of those for parenteral, subcutaneous, intravenous, intramuscular, intraarterial, intrathecal, intratumoral, or interperitoneal administration. More than one route can be used to administer the inventive nanoparticles, and in certain instances, a particular route can provide a more immediate and more effective response than another route. Preferably, the inventive nanoparticles are administered by injection, e.g., intravenously.

For purposes of the invention, the amount or dose (e.g., numbers of nanoparticles) of the inventive nanoparticles administered should be sufficient to effect, e.g., a therapeutic or prophylactic response, in the subject or animal over a reasonable time frame. For example, the dose of the inventive nanoparticulars should be sufficient to reduce the expression of a target gene or detect, treat or prevent disease (e.g., cancer or a viral disease) in a period of from about 2 hours or longer, e.g., 12 to 24 or more hours, from the time of administration. In certain embodiments, the time period could be even longer. The dose will be determined by the efficacy of the particular inventive nanoparticles, the particular functional moiety (or moieties) attached to the nanoparticles, and the condition of the animal (e.g., human), as well as the body weight of the animal (e.g., human) to be treated.

Human dosage amounts can initially be determined by extrapolating from the amount of nanoparticles used in mice, as a skilled artisan recognizes it is routine in the art to modify the dosage for humans compared to animal models. In certain embodiments it is envisioned that the dosage may vary from between about 1 mg RNA nanostructure/Kg body weight to about 5000 mg RNA nanostructure/Kg body weight; or from about 5 mg/Kg body weight to about 4000 mg/Kg body weight; or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other embodiments, this dose may be about 1, about 5, about 10, about 25, about 50, about 75, about 100, about 150, about 200, about 250, about 300, about 350, about 400, about 450, about 500, about 550, about 600, about 650, about 700, about 750, about 800, about 850, about 900, about 950, about 1000, about 1050, about 1100, about 1150, about 1200, about 1250, about 1300, about 1350, about 1400, about 1450, about 1500, about 1600, about 1700, about 1800, about 1900, about 2000, about 2500, about 3000, about 3500, about 4000, about 4500, about 5000 mg/Kg body weight, or a range defined by any two of the foregoing values. In other embodiments, it is envisaged that higher does may be used, such doses may be in the range of about 5 mg RNA nanoparticles/Kg body to about 20 mg RNA nanoparticles/Kg body. In other embodiments, the doses may be about 8, about 10, about 12, about 14, about 16 or about 18 mg/Kg body weight. Of course, this dosage amount may be adjusted upward or downward, as is routinely done in such treatment protocols, depending on the results of the initial clinical trials and the needs of a particular patient.

The dose of the inventive RNA nanoparticles also will be determined by the existence, nature and extent of any adverse side effects that might accompany the administration of a particular inventive RNA nanoparticles. Typically, the attending physician will decide the dosage of the inventive RNA nanoparticles with which to treat each individual patient, taking into consideration a variety of factors, such as age, body weight, general health, diet, sex, inventive RNA nanoparticles to be administered, route of administration, and the severity of the disease, e.g., cancer being treated.

It is contemplated that the inventive RNA nanoparticles may be useful for modulating the expression of a target gene in a mammal. In this regard, an embodiment of the invention provides a method of modulating the expression of a target gene in a mammal, the method comprising administering any of the RNA nanoparticles described herein or any of the pharmaceutical compositions described herein in an amount effective to modulate the target gene. In an embodiment of the invention, the expression of the target gene is modulated by increasing the expression of the target gene in the mammal to which the RNA nanostructure is administered as compared to the expression of the target gene in a mammal which has not been administered the RNA nanostructure. In another embodiment of the invention, the expression of the target gene is modulated by decreasing or eliminating the expression of the target gene in the mammal to which the RNA nanoparticles is administered as compared to the expression of the target gene in a mammal which has not been administered the RNA nanostructure. The quantity of expression of a target gene may be assayed by methods known in the art.

It is also contemplated that the inventive RNA nanoparticles may be useful for treating or preventing a disease in a mammal. In this regard, an embodiment of the invention provides a method of treating or preventing a disease in a mammal, the method comprising administering any of the RNA nanoparticles described herein or any of the pharmaceutical compositions described herein in an amount effective to treat or prevent the disease in the mammal.

In an embodiment of the invention, the disease is cancer. The cancer can be any cancer, including any of sarcomas (e.g., synovial sarcoma, osteogenic sarcoma, leiomyosarcoma uteri, and alveolar rhabdomyosarcoma), lymphomas (e.g., Hodgkin lymphoma and non-Hodgkin lymphoma), hepatocellular carcinoma, glioma, head-neck cancer, acute lymphocytic cancer, acute myeloid leukemia, bone cancer, brain cancer, breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer (e.g., colon carcinoma), esophageal cancer, cervical cancer, gastrointestinal carcinoid tumor, hypopharynx cancer, larynx cancer, liver cancer, lung cancer, malignant mesothelioma, melanoma, multiple myeloma, nasopharynx cancer, ovarian cancer, pancreatic cancer, peritoneum, omentum, and mesentery cancer, pharynx cancer, prostate cancer, rectal cancer, renal cancer, small intestine cancer, soft tissue cancer, stomach cancer, testicular cancer, thyroid cancer, ureter cancer, and urinary bladder cancer. In an embodiment of the invention, the cancer is breast cancer.

In an embodiment of the invention, the disease is a viral disease. The viral disease may be caused by any virus. In an embodiment of the invention, the viral disease is caused by a virus selected from the group consisting of herpes viruses, pox viruses, hepadnaviruses, papilloma viruses, adenoviruses, coronoviruses, orthomyxoviruses, paramyxoviruses, flaviviruses, and caliciviruses. In an embodiment, the viral disease is caused by a virus selected from the group consisting of respiratory syncytial virus (RSV), influenza virus, herpes simplex virus, Epstein-Barr virus, varicella virus, cytomegalovirus, hepatitis A virus, hepatitis B virus, hepatitis C virus, human T-lymphotropic virus, calicivirus, adenovirus, human immunodeficiency virus, and Arena virus.

The viral disease may be any viral disease affecting any part of the body. In an embodiment of the invention, the viral disease is selected from the group consisting of influenza, pneumonia, herpes, hepatitis, hepatitis A, hepatitis B, hepatitis C, chronic fatigue syndrome, sudden acute respiratory syndrome (SARS), gastroenteritis, enteritis, carditis, encephalitis, bronchiolitis, respiratory papillomatosis, meningitis, and mononucleosis.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the inventive methods can provide any amount of any level of treatment or prevention of a disease in a mammal. Furthermore, the treatment or prevention provided by the inventive method can include treatment or prevention of one or more conditions or symptoms of the viral disease, being treated or prevented. Also, for purposes herein, “prevention” can encompass delaying the onset of the disease, or a symptom or condition thereof.

In an embodiment, the patient referred to the inventive methods is a mammal. As used herein, the term “mammal” refers to any mammal, including, but not limited to, mammals of the order Rodentia, such as mice and hamsters, and mammals of the order Logomorpha, such as rabbits. It is preferred that the mammals are from the order Carnivora, including Felines (cats) and Canines (dogs). It is more preferred that the mammals are from the order Artiodactyla, including Bovines (cows) and Swines (pigs) or of the order Perssodactyla, including Equines (horses). It is most preferred that the mammals are of the order Primates, Ceboids, or Simoids (monkeys) or of the order Anthropoids (humans and apes). An especially preferred mammal is the human.

The following examples further illustrate the invention but, of course, should not be construed as in any way limiting its scope.

### EXAMPLES

The following materials and methods were employed in the experiments described in Examples 1 through 6, below.

### Computational Considerations and RNA/DNA Hybrid Construct Design

The computational folding of individual strands and assembly of DNA/RNA constructs was assessed using HyperFold (see Bindewald et al., Nano Lett., 16: 1726-1735 (2016)), a nucleic acid structure prediction algorithm capable of predicting multi-strand assembles from combinations of RNA and DNA strands. All folding predictions were performed at strand concentrations of 1 μM at 37° C. The program RiboSketch was used to visualize the resulting secondary structure predictions (see Lu, et al., Bioinformatics, 34(24): 4297-99 (2018)).

### Oligonucleotide Synthesis and Purification

The DNA and RNA oligonucleotides used to assemble the conditional RNA/DNA constructs, including those that were fluorescently labeled, were purchased from Integrated DNA Technologies (IDT, Coralville, Iowa) and reconstituted in nuclease free water (Quality Biological, Gaithersberg, Md.) for use. All fluorescently labeled oligonucleotides were purchased from IDT. Ten nmol quantities of the oligonucleotides were purified as needed by denaturing PAGE. Ten nmol quantities were mixed with 100 uL urea loading buffer (6 M urea, 20 mM EDTA, 10% glycerol, 0.05% bromophenol blue) and heated to 90° C. for 2 minutes prior to loading on an 8% or 10% acrylamide denaturing gel (1×TBE buffer [89 mM Tris, 89 mM boric acid, 2 mM ethylenediaminetetraacetic acid (EDTA)], 6 M Urea) for purification. Following electrophoresis, bands were cut from the gel and eluted in an elution buffer (10 mM Tris pH 7.5, 200 mM NaCl, 0.5 mM EDTA) overnight at 4° C. while shaken at 850 rpm. Eluted oligonucleotides were ethanol precipitated and reconstituted in nuclease-free water.

RNA trigger oligonucleotides were either purchased from IDT or prepared from an in vitro runoff transcription using T7 RNA polymerase. DNA templates for transcription were amplified by polymerase chain reaction (PCR) using primers purchased form IDT. PCR was performed using MYTAQ™ 2× mix (Bioline, London, UK) and purified using DNA CLEAN & CONCENTRATOR™ (Zymo Research, Irvine, Calif.). Transcription was performed in 10 mM Tris pH 7.0 containing 6 mM MgCl₂, 0.5 mM MnCl₂, 2.5 mM each NTP, 0.01 u/μL inorganic pyrophosphatase, 2 mM dithiothreitol, and 2 mM spermidine. Approximately 50 pmol of DNA template was added to the transcription mix along with an in-house produced T7 RNA polymerase and incubated at 37° C. for 4 hours. Transcription was terminated by addition of DNase I (New England Biolabs, Ipswich, Mass.) for 30 minutes. The transcription mix was combined with ½ volume of urea loading buffer and heated at 90° C. for 2 minutes before purification by denaturing PAGE and precipitation as described above.

### RNA/DNA Construct Assembly

Conditional RNA/DNA constructs were assembled using equimolar concentrations of their component strands. Strands were combined in water, heated to 90° C. for 1.5 minutes, then immediately placed on a 37° C. heat block for 5 minutes. After this, samples were briefly spun in a tabletop centrifuge to collect condensed solvent and assembly buffer was added to a final 1× concentration of 2 mM Mg(OAc)₂, 50 mM KCl, 1×TB (89 mM Tirs, 89 mM boric acid, pH 8.2). The assembly was then incubated an additional 25 minutes at 37° C. Control dsRNA duplexes and RNA trigger molecules were assembled/folded using the same protocol.

### Non-Denaturing PAGE Analysis of Conditional Oligonucleotide Release

Assembled constructs were examined for their ability to regulate conditional oligonucleotide release in the presence and absence of specific RNA trigger molecules. All constructs and triggers were initially prepared separately in 1× assembly buffer. From these bulk individual assemblies, various construct/trigger combinations were combined and incubated at 37° C. for either 30, 90 or 180 minutes. Individual controls were prepared from the same bulk assemblies and subjected to identical incubation conditions. Generally, the conditional constructs were present at a final concentration of 500 nM. In the case of the beacon switch and adjacent targeting hybrids, RNA triggers were present at 1× concentration relative to the conditional constructs. For inducible and repressible hybrid systems, the RNA triggers were generally present at 2×-3× concentrations, as indicated in the text. Following this incubation samples were transferred to ice, combined with ⅕ volume of loading buffer (1× assembly buffer, 50% glycerol) and were loaded on non-denaturing PAGE gels (8-12% 19:1 acrylamide/bis-acrylamide, 2 mM Mg(OAc)₂, 1×TB). Electrophoresis was generally performed at 6 W for 2-3 hr at 10° C. Acrylamide concentrations and duration of electrophoresis was optimized on a case by case basis to achieve the necessarily separation of species. In some instances, gels were subjected to total nucleic acid staining with ethidium bromide. In other instances, an individual molecule within a construct was fluorescently labeled (˜10% of total molecules used in an assembly). In both cases, gels were imaged using a Typhoon Trio variable mode imager (GE Healthcare, Little Chalfont, England) using appropriate excitation and emission filters. The amount of fluorescently labeled dsRNA output released from conditional systems was quantitated using IMAGEQUANT 5.1 software (GE Healthcare). Unless otherwise noted, the fraction of dsRNA released for a given sample is reported as the ratio of fluorescence observed in the released dsRNA band to the total amount of fluorescence observed for the entire lane. Statistical significance between populations was determined by two-tailed Student's t-Test performed using values from three distinct replicate experiments.

### Analysis of RNA/DNA Strand Exchange by FRET

RNA/DNA strand exchange between cognate partners of inducible and repressible hybrid systems were examined by FRET. Cognate hybrids were assembled separately, and pre-warmed to 37° C. Hybrids were combined and added to the cuvette, at which point the RNA trigger molecule was spiked in, if appropriate. The cuvette was immediately placed in a FLUOROMAX-3 fluorimeter (Horiba Ltd., Kyoto, Japan) at 37° C. and measurement was started. For FRET experiments where a fluorescence spectrum was measured at a given time point the sense hybrid was assembled with an RNA sense strand containing a 3′ 6-FAM donor fluorophore, while the antisense hybrid was assembled with an RNA antisense strand possessing a 5′ ALEXAFLUOR546 acceptor fluorophore (Thermo Fisher Scientific, Waltham, Mass.). Hybrids were prepared to a final concentration of 250 mM and the trigger molecule was in three-fold molar excess, when present. Excitation was performed at 475 nm and emission measured between 480-620 nm at 1 nm increments using 0.5 s integration times and 2 nm slit widths.

For FRET experiments where time courses were recorded the 6-FAM donor fluorophore on the RNA sense strand was replaced with ALEXAFLUOR488 (Thermo Fisher Scientific). Hybrid and trigger concentration mirrored the conditions of analogous non-denaturing PAGE experiments, with hybrids at a final concentration of 500 nM, and trigger concentrations in 2-3 fold molar excess, as indicated. Measurements were recorded every 60 seconds using excitation at 475 nm and emission was measured at 515 nm and 565 nm, using a signal integration time of 0.5 s and slit widths of 2 nm. Observed rate constants (k_{obs}) were obtained by fitting the decrease in measured ALEXAFLUOR488 donor fluorescence as a function of time to the equation y=y₀+Ae^{-k*t} for single exponential decay.

### Adjacent-Targeting Hybrids

The system was designed to release a 25/27 Dicer substrate siRNA (DsiRNA) product from a sense and antisense RNA/DNA hybrid pair following interaction with a fragment of the CTGF mRNA. The sense hybrid (sH_{DOWN}) contains a 5′ DNA toehold designed to bind a sequence region of the CTGF trigger downstream of the binding site for the antisense hybrid's (aH_{UP}) 3′ DNA toehold. The basic aH_{UP} and sH_{DOWN} hybrid constructs were designed with 12 nucleotide (nt) toeholds emanating from the RNA/DNA hybrid duplex region. The upstream and downstream regions for toehold binding were separated by only a single nucleotide in the RNA trigger. This is designed to position the RNA/DNA hybrid regions next to one another in 3D space, while the single nucleotide gap between the trigger-bound toeholds provides some steric flexibility. Additional DNA nucleotides were eventually inserted between the RNA/DNA hybrid regions and the toeholds. These DNA nucleotides were complementary between cognate sH_{Down} and aH_{UP} hybrid pairs, and acted to as a nucleation site for the strand exchange reaction.

### CTGF-Induced Hybrids

The system was designed to release a 25/27 Dicer substrate siRNA (DsiRNA) product from a sense and antisense RNA/DNA hybrid pair following interaction with a fragment of the CTGF mRNA. The sense hybrid (sH_{\hat{ }CTGF}) contained a DNA strand that was complementary to the sense RNA. The DNA strand was extended in the 5′ direction to encode a sequence that formed the diagnostic domain. A structured DNA hairpin was designed immediately 5′ adjacent to the RNA/DNA hybrid region. Initially, this hairpin contained a 12 base pair stem and 8 nucleotide loop (sH_{\hat{ }CTGF.12/8}), but multiple variants with differences in the stem length and loop size were ultimately constructed. Flanking the hairpin on the 5′ side is a diagnostic toehold 20 nucleotide in length for most sH_{\hat{ }CTGF} constructs. The diagnostic toehold of sH_{\hat{ }CTGF.20/8} was reduced in length to 16 nucleotides to keep the total length of the DNA strand from exceeding 90 nucleotides. For sH_{\hat{ }CTGF} hybrids with a 12 base pair hairpin stem the diagnostic toehold, 5′ side of the hairpin stem, and the first four nucleotides of the hairpin loop were designed to be complementary to a continuous region of the CTGF mRNA. For sH_{\hat{ }CTGF} hybrids with 16 base pair or 20 base pair hairpin stems complementarity to the CTGF trigger extended up the entirety of the hairpin stem, but did not include any loop nucleotides. The exchange toehold for sH_{\hat{ }CTGF} hybrids was encoded in the DNA sequence immediately 5′ to the region hybridized to the sense RNA strand, and were ultimately sequestered to serve at the 3′ side of the DNA hairpin stem in the initially folded structure. The cognate antisense hybrids (aH_{\hat{ }CTGF-cgnt}) contained a DNA strand that hybridized to the antisense RNA strand at it 5′ end. From this RNA/DNA hybrid duplex region the 3′ end of the DNA strand was extended to encode the complementary exchange toehold. Two variants were created. One contained a 12 nucleotide toehold, while the other contained a 16 nucleotide toehold.

### KRAS-Repressed Hybrids

The system was designed to release a 25/27 Dicer substrate siRNA (DsiRNA) product from a sense and antisense RNA/DNA hybrid pair in the absence of any interaction with a fragment of the KRAS mRNA. The antisense hybrid (aH_{vKRAS}) contained a DNA strand that was designed at its 5′ end to be complementary to the antisense RNA, creating the RNA/DNA hybrid region. Immediately adjacent to the hybrid region, the DNA strand encodes the 12 nucleotide exchange toehold followed by a DNA hairpin. The DNA hairpin contains a 14 base pair stem and 12 nucleotide loop. The 12 nucleotide hairpin loop is designed to be complementary to the 12 nucleotide exchange toehold adjacent to the base of the hairpin stem, which can fold to form a less stable alternative hairpin. These complementary loop and toehold sequences that defined the stem of the alternative hairpin were designed to be AU-rich in order to initially favor formation of the primary 14 base pair hairpin. This pair of alternative hairpin structures provides the mechanism to repress strand exchange. An 11 nucleotide single-strand diagnostic toehold is incorporated that exits directly from the 3′ side of the 14 base pair hairpin. The diagnostic toehold and the adjacent 3′ side of the hairpin are complementary to a continuous region of the KRAS mRNA. Binding of the KRAS trigger is designed to unzip the primary hairpin and induce a conformational change that results in formation of the alternative hairpin, sequestering the exchange toehold within its stem, and ultimately represses dsRNA release. The cognate sense hybrid (sH_{vKRAS-cgnt}) contained a DNA strand that contained a sequence at its 3′ to hybridized to the sense RNA strand. From this RNA/DNA hybrid duplex region the 5′ end of the DNA strand was extended to encode the complementary 12 exchange toehold.

### Sequences and Assemblies Used

*Sequences are indicated as either RNA or DNA

### Beacon-Derived Switch (KRAS Triggered)

### DNA Diagnostic Strand:

(SEQ ID NO: 1)
TTTGTTCGTTTCATTGCACTGTACTCCTCTTGGCTCGCTGTGA
RNA output strand (anti-miR 375):
(SEQ ID NO: 2)
UCACGCGAGCCGAACGAACAAA
Adjacent-targeting RNA/DNA hybrids (CTGF triggered)
Obp aH_{UP}:
(SEQ ID NO: 3)
aRNA CGGUGGUGCAGAUGAACUUCAGGGUCA
(SEQ ID NO: 4)
a′DNA tgaccctgaagttcatctgcaccaccgagttgtaatggc
Obp sH_{DOWN}:
(SEQ ID NO: 5)
sRNA ACCCUGAAGUUCAUCUGCACCACCG
(SEQ ID NO: 6)
s′DNA ttgtctccgggacggtggtgcagatgaacttcagggt
+1bp aH_{UP}:
(SEQ ID NO: 7)
aRNA CGGUGGUGCAGAUGAACUUCAGGGUCA
(SEQ ID NO: 8)
a′DNA tgaccctgaagttcatctgcaccaccggagttgtaatggc
+1bp sH_{DOWN}:
(SEQ ID NO: 9)
sRNA ACCCUGAAGUUCAUCUGCACCACCG
(SEQ ID NO: 10)
s′DNA ttgtctccgggaccggtggtgcagatgaacttcagggt
+2bp aH_{UP}:
(SEQ ID NO: 11)
aRNA CGGUGGUGCAGAUGAACUUCAGGGUCA
(SEQ ID NO: 12)
a′DNA tgaccctgaagttcatctgcaccaccgcgagttgtaatggc
+2bp sH_{DOWN}:
(SEQ ID NO: 13)
sRNA ACCCUGAAGUUCAUCUGCACCACCG
(SEQ ID NO: 14)
s′DNA ttgtctccgggacgcggtggtgcagatgaacttcagggt
+3bp aH_{UP}:
(SEQ ID NO: 15)
aRNA CGGUGGUGCAGAUGAACUUCAGGGUCA
(SEQ ID NO: 16)
a′DNA tgaccctgaagttcatctgcaccaccggcgagttgtaatggc
+3bp sH_{DOWN}:
(SEQ ID NO: 17)
sRNA ACCCUGAAGUUCAUCUGCACCACCG
(SEQ ID NO: 18)
s′DNA ttgtctccgggacgccggtggtgcagatgaacttcagggt
+4bp aH_{UP}:
(SEQ ID NO: 19)
aRNA CGGUGGUGCAGAUGAACUUCAGGGUCA
(SEQ ID NO: 20)
a′DNA tgaccctgaagttcatctgcaccaccgggcgagttgtaatggc
+4bp sH_{DOWN}:
(SEQ ID NO: 21)
sRNA ACCCUGAAGUUCAUCUGCACCACCG
(SEQ ID NO: 22)
s′DNA ttgtctccgggacgcccggtggtgcagatgaacttcagggt
Inducible activation hybrids (CTGF triggered) [symbol]
:
(SEQ ID NO: 23)
aRNA CGGUGGUGCAGAUGAACUUCAGGGUCA
(SEQ ID NO: 24)
a′DNA tgaccctgaagttcatctgcaccaccg aagatgtcattg [symbol]
:
(SEQ ID NO: 25)
aRNA CGGUGGUGCAGAUGAACUUCAGGGUCA
(SEQ ID NO: 26)
a′DNA tgaccctgaagttcatctgcaccaccg aagatgtcattgtctc [symbol]
:
(SEQ ID NO: 27)
sRNA ACCCUGAAGUUCAUCUGCACCACCG
(SEQ ID NO: 28)
s′DNA tcctgtagtacagcgattca aagatgtcattg tctcaacc caatgacatctt
cggtggtgcagatgaacttcagggtca [symbol]
:
(SEQ ID NO : 29)
sRNA ACCCUGAAGUUCAUCUGCACCACCG
(SEQ ID NO: 30)
s′DNA tcctgtagtacagcgattca aagatgtcattg tctcaacaccat
caatgacatctt cggtggtgcagatgaacttcagggtca [symbol]
:
(SEQ ID NO: 31)
sRNA ACCCUGAAGUUCAUCUGCACCACCG
(SEQ ID NO: 32)
s′DNA tcctgtagtacagcgattca aagatgtcattgtctc aagcggac
gagacaatgacatctt cggtggtgcagatgaacttcagggtca [symbol]
:
(SEQ ID NO: 33)
sRNA ACCCUGAAGUUCAUCUGCACCACCG
(SEQ ID NO: 34)
s′DNA tagtacagcgattca aagatgtcattgtctccggg aagcggac
cccggagacaatgacatctt cggtggtgcagatgaacttcagggtca
3-piece inducible activation hybrid (CTGF triggered): [symbol]
:
(SEQ ID NO: 35)
sRNA ACCCUGAAGUUCAUCUGCACCACCG
(SEQ ID NO: 36)
s′DNA1 TAGTACAGCGATTCA AAGATGTCATTGTCTCCGGG
(SEQ ID NO: 37)
s′DNA2 CCCGGAGACAATGACATCTT
CGGTGGTGCAGATGAACTTCAGGGTCA
Trigger repressible RNA/DNA hybrids (CTGF or KRAS triggered) [symbol]
:
(SEQ ID NO: 38)
aRNA CGGUGGUGCAGAUGAACUUCAGGGUCA
(SEQ ID NO: 39)
a′DNA TGACCCTGAAGTTCATCTGCACCACCG AAGATGTCATTG
GCAATGAGGGACCA CAATGACATCTT TGGTCCCTCATTGC
ACTGTACTCCT
aH_{vCTGF.cgnt}:
(SEQ ID NO: 40)
aRNA CGGUGGUGCAGAUGAACUUCAGGGUCA
(SEQ ID NO: 41)
a′DNA tgaccctgaagttcatctgcaccaccg ACTGTAATGCTA [symbol]
:
(SEQ ID NO: 42)
sRNA ACCCUGAAGUUCAUCUGCACCACCG
(SEQ ID NO: 43)
s′DNA CAATGACATCTT cggtggtgcagatgaacttcagggt [symbol]
:
(SEQ ID NO: 44)
sRNA ACCCUGAAGUUCAUCUGCACCACCG
(SEQ ID NO: 45)
s′DNA AGATGTCATTGTC TCCGGGACAGTTGT ACTGTAATGCTA
ACAACTGTCCCGGA TAGCATTACAGT
CGGTGGTGCAGATGAACTTCAGGGT
3-piece trigger repressible hybrids (KRAS triggered): [symbol]
:
(SEQ ID NO: 46)
aRNA CGGUGGUGCAGAUGAACUUCAGGGUCA
(SEQ ID NO: 47)
a′DNA1TGACCCTGAAGTTCATCTGCACCACCG AAGATGTCATTG
GCAATGAGGGACCA CAATGACATCTT
(SEQ ID NO: 48)
a′DNA2TGGTCCCTCATTGC ACTGTACTCCT [symbol]
:
(SEQ ID NO: 49)
aRNA CGGUGGUGCAGAUGAACUUCAGGGUCA
a′DNA1TGACCCTGAAGTTCATCTGCACCACCG AAGATGTCATTG
(SEQ ID NO: 50)
GCAATGAGGGACCA CAATGACATCTT TGGT
(SEQ ID NO: 51)
a′DNA2CCCTCATTGC ACTGTACTCCT [symbol]
:
(SEQ ID NO: 52)
aRNA CGGUGGUGCAGAUGAACUUCAGGGUCA
(SEQ ID NO: 53)
a′DNA1TGACCCTGAAGTTCATCTGCACCACCG AAGATGTCATTG
GCAATGAGGGACCA CAATGACATCTT TGGTCC
(SEQ ID NO: 54)
a′DNA2CTCATTGC ACTGTACTCCT [symbol]
:
(SEQ ID NO: 55)
aRNA CGGUGGUGCAGAUGAACUUCAGGGUCA
(SEQ ID NO: 56)
a′DNA1 TGACCCTGAAGTTCATCTGCACCACCG AAGATGTCATTG
GCAATGAGGGACCA CAATGACATCTT TGGTCCCT
(SEQ ID NO: 57)
a′DNA2 CATTGC ACTGTACTCCT

### RNA Trigger Sequences

*SEQ ID NOs: 58 and 60 were added to the 5′ end of the CTGF and KRAS sequences, respectively, for the purpose of in vitro transcription and are not present in the endogenous sequence from which the truncated mRNA fragments were derived.

CTGF:
(SEQ ID NO: 58)
ggga
(SEQ ID NO: 59)
AAGACCUGUGCCUGCCAUUACAACUGUCCCGGAGACAAUGACAUCUU
UGAAUCGCUGUACUACAGGAAGAUGUACGG
KRAS:
(SEQ ID NO: 60)
ggg
(SEQ ID NO: 61)
CUCGACACAGCAGGUCAAGAGGAGUACAGUGCAAUGAGGGACCAGUA
CAUGAGGACUGGG
Random sequence:
(SEQ ID NO: 63)
GGCAACUUUGAUCCCUCGGUUUAGCGCCGGCCUUUUCUCCCACACUU
UCACG

### Example 1

This example demonstrates that beacon-derived conditional switches according to embodiments of the invention release single-stranded oligonucleotides in the presence of an RNA target.

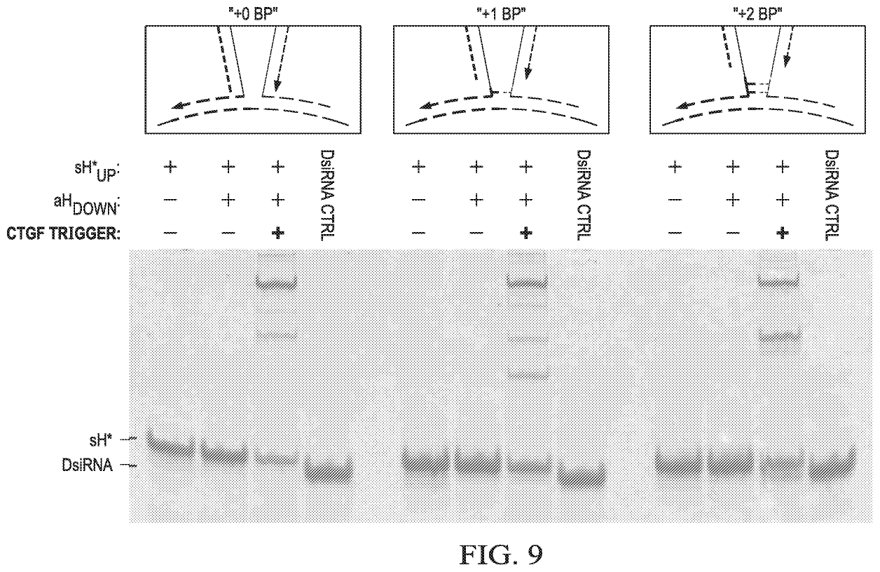

A beacon switch was designed to respond to a fragment of the KRAS mRNA (SEQ ID NO:61) as a trigger (i.e., KRAS trigger) and release an RNA antagomir output strand in a conditional fashion. Analysis of beacon switch assembly and conditional output release was performed by non-denaturing PAGE (see FIG. 2C). Assembly between the diagnostic strand and output strand to form the beacon switch was extremely efficient as determined by non-denaturing PAGE and total nucleic acid staining, with only trace amounts of the single-stranded output strands observed after assembly. Co-incubation of the assembled beacon switch with the KRAS trigger at 37° C. results in the release of the output strand and the appearance of a band corresponding to the expected waste product. A higher migrating band also appears in this lane, which was a trinary molecular complex of the assembled beacon switch bound to the trigger. The amount of output strand observed to be released was likely a lower limit of the amount of single-stranded oligo that was actually newly accessible following interaction with the trigger RNA. This was because only one end of the single-stranded output needs to be released by the diagnostic strand to allow complete hybridization with the trigger oligonucleotide (see FIG. 7). However, even a partially released single-stranded output oligo should be accessible to hybridize to a target RNA and still be able to perform its intended regulatory function.

Traditional molecular beacons act as a unimolecular diagnostic tool, giving a fluorescent output signal that changes as a result of the presence of a specific oligonucleotide trigger (FIG. 2A, Tyagi et al., Nat. Biotechnol., 14: 303-308 (1996)). Rather than use fluorescence as an output signal, the beacon system was re-engineered as a bimolecular switch construct that was able to release a single-stranded oligonucleotide upon recognition of a specific trigger sequence. Whereas traditional molecular beacons contain complementary regions at the 5′ and 3′ ends resulting in a hairpin structure (FIG. 2A), the present beacon switch was designed such that the output oligonucleotide was complementary across its length to the 5′ and 3′ ends of the diagnostic strand, generating a structure that resembles the shape of a horseshoe (FIG. 2B). The diagnostic strand contains a large loop that was complementary to the trigger and serves as an internal toehold. Hybridization between the internal toehold and the trigger RNA acts as a thermodynamic driver that was intended to disrupt the pairing between the output strand and the diagnostic strand, resulting in the release of the single-stranded output.

Since the internal toehold of the diagnostic strand does not need to overlap with the 5′ and 3′ regions that are bound to the output oligonucleotide, essentially any set of trigger and target sequences can be implemented. The single-stranded output of the beacon switch could be composed of RNA or DNA depending on the desired function of the output strand. This conditional system could find application in instances where an irregular or diseased cellular state can be identified by a high copy number of a specific endogenous RNA, and the use of an AON, antagomir or other short single-stranded RNA would have significant impact on rectifying the irregular state or inducing cell death.

### Example 2

This example demonstrates that strand exchange between RNA/DNA hybrid duplexes according to embodiments of the invention can be facilitated by toeholds that target adjacent sequence regions of an RNA target.

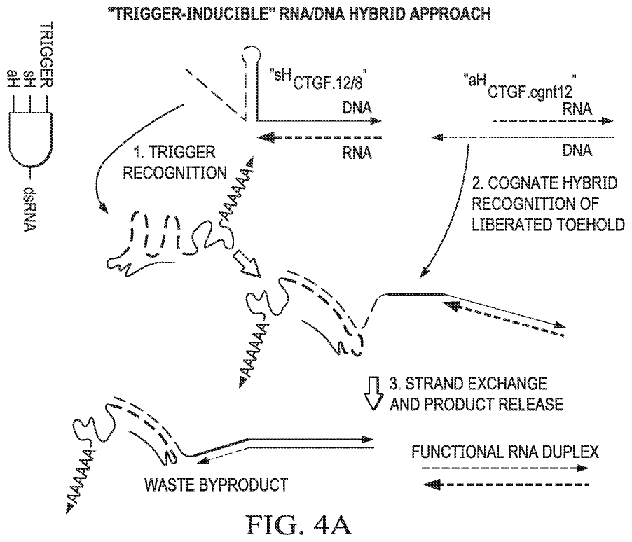

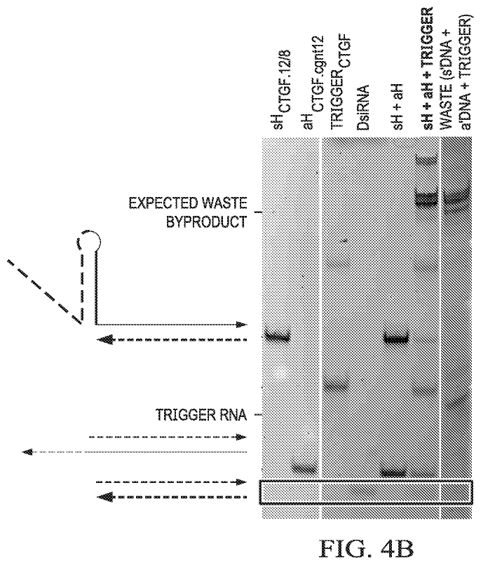

Cognate RNA/DNA hybrid pairs were previously designed that harbor split functional RNAs, devised to release a recombined functional dsRNA through recognition of complementary single stranded toeholds (FIG. 3A; Afonin et al., Nat. Nanotechnol., 8: 296-304 (2013); Afonin et al., Nucleic Acids Res., 42: 2085-2097 (2014); and Afonin et al., Nano Lett., 16: 1746-1753 (2016)). The separated single strands composing the functional duplex can be referred to as the sense strand and the antisense strand, and each of these RNA strands were annealed to a complementary DNA oligonucleotide. These assembled RNA/DNA hybrids are denoted as the sense hybrid (sH) and the antisense hybrid (all), respectively. The “traditional” approach to cognate hybrid design utilized complementary single stranded toeholds emanating from sH and aH, with hybridization of these toeholds to one another initiating RNA/DNA strand exchange. Here, the toeholds are redesigned to be complementary to adjacent regions of an RNA target sequence, rather than complementary to one another. As the toeholds can no longer drive strand exchange by hybridization to one another, release of the dsRNA product was conditional on the presence of the RNA target molecule.

In this “adjacent targeting” incarnation of the RNA/DNA hybrid system, a fragment of the CTGF (SEQ ID NO:59) mRNA was used as the RNA target sequence, acting as a template for DNA toehold binding which in turn initiates strand exchange (FIG. 3B). Since the antisense hybrid binds upstream on the RNA target, it was termed aH_{UP}. Similarly, the sense hybrid was referred to as sH_{DOWN}. Binding of the cognate hybrid pair to the trigger RNA positions the two RNA/DNA hybrid regions adjacent to one another in space. The close proximity of the trigger-bound cognate hybrids will induce strand exchange through progressive hybridization of the trigger-bound DNA strands to one another, forming a three-way junction with the RNA target, and leading to formation and release of a dsRNA product. Like the beacon-derived switch, this activatable RNA/DNA hybrid system could find use in instances where a cell population of interest can be distinguished by the high relative expression level of an endogenous RNA. However, this RNA/DNA hybrid system (and those that follow) could be of use in cases where conditional generation of a double-stranded RNA was desirable, which could take the form of an RNA interference substrate, saRNA, aptamer, or another functionally relevant dsRNA. In this instance, the dsRNA product was designed as a 25/27-mer DsiRNA.

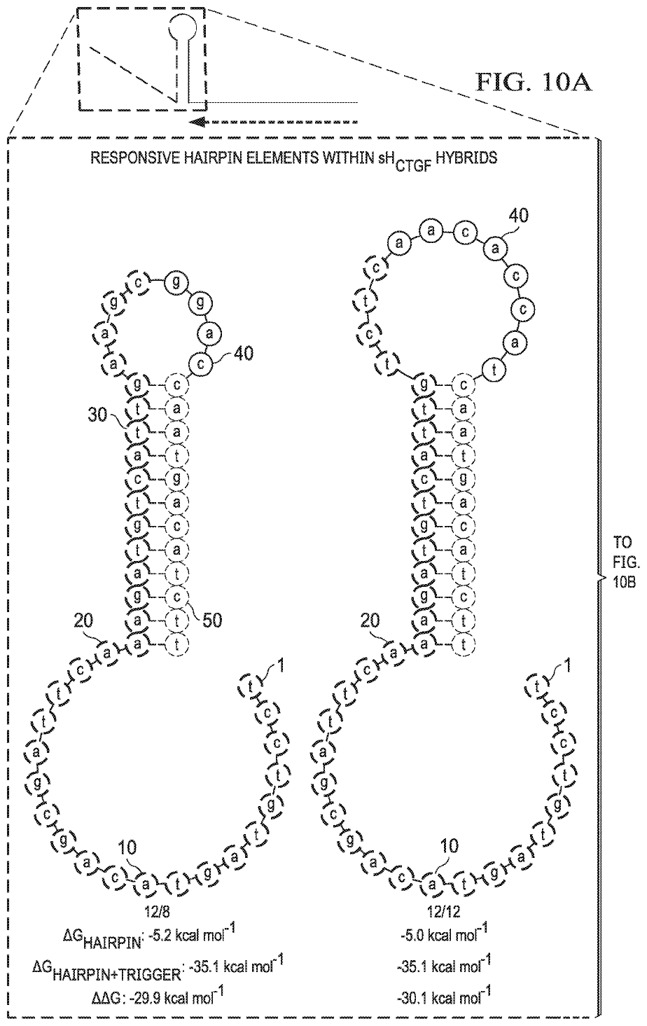

Formation of dsRNA product was visualized by non-denaturing PAGE. The initial aH_{UP}/sH_{DOWN} cognate pair did not induce strand exchange and dsRNA release when co-incubated with the CTGF trigger for 180 minutes (FIG. 3C, “0 bp”). In the presence of the RNA target a large fraction of the hybrid constructs appear to be stuck in an intermediate complex displaying slow electrophoretic mobility. Presumably, this observed band corresponds to a state in which both RNA/DNA hybrids are bound to the trigger through their respective toeholds, but strand exchange in not stimulated. Despite no observed dsRNA release from this system, the strand exchange reaction was predicted to be thermodynamically favored (FIG. 8). In an attempt to provide a greater driving force for strand exchange, additional sets of cognate hybrids pairs were designed in which additional complementary DNA nucleotides were inserted between the toehold region and the RNA/DNA hybrid region of each hybrid construct. These complementary nucleotides were inserted to essentially serve as a nucleation site for strand exchange between the cognate partners once bound to the RNA target. In total, four additional hybrid pairs were designed which contained between 1 and 4 additional base pairs to seed the strand exchange (FIG. 3C).

Increasing the number of complementary DNA base pairs inserted immediately prior to the RNA/DNA hybrid regions resulted in increased DsiRNA release. Insertion of at least 2 DNA base pairs was needed to observe significant increases in DsiRNA release in the presence of the trigger RNA, as compared to background in the absence of the trigger after three hours (Table 2). Insertion of 3 base pairs appears to be enough to achieve close to the maximal degree of product duplex release, as increasing to 4 inserted base pairs results in negligible further increases in DsiRNA release after 180 minutes. However, the gel electrophoresis experiments suggest that insertion of additional bae pairs does speed up the rate at which this plateau of apparent maximal possible product release was reached, as the +4 base pair releases significantly more dsRNA after 30 minutes than the +3 base pair system, and likewise the +3 base pair system shows greater release than the +2 base pair hybrid pair (Table 3, statistical significance in the difference of dsRNA fraction released from differing adjacent targeting hybrid pairs at a single time point, in presence or absence of the CTGF RNA trigger. P-values indicated as follows: not significant (ns) if >0.05; * if <0.05; ** if <0.01; *** if <0.001). Despite the +3 base pair and +4 base pair hybrid pairs eventually reaching a similar level of dsRNA release after three hours, their differences in the fraction of dsRNA released at early time points suggests that the initiation of strand exchange within the adjacent targeting system may be impeded by slow kinetics. Interestingly, despite these systems containing complementary DNA nucleotides that could potentially serve as toeholds to promote strand exchange in the absence of the trigger RNA, increasing the number of inserted seed base pairs up to four did not result in significant differences in the degree of non-triggered dsRNA release when co-incubated over the longest duration examined (Table 1; and Table 2, statistical significance in the difference of dsRNA fraction released from individual adjacent targeting hybrid pairs at various time points, in presence or absence of the CTGF RNA trigger. P-values indicated as follows: not significant (ns) if >0.05; * if <0.05; ** if <0.01; *** if <0.001.) In Table 1, the average fraction of dsRNA release is reported in presence and absence of CTGF trigger, at each of three time intervals examined. An efficiency score metric is determined for each hybrid pair at a given time point, with larger score indicating better efficiency of conditional dsRNA release. The efficacy score takes into account both the fraction of dsRNA released and the signal to noise ratio. It was calculated as (fraction of triggered release)*(fraction triggered release/fraction non-triggered release). The hybrid pairing that yielded greatest efficiency score at each of the three time intervals examined was sH_{\hat{ }CTGF.20/8} and aH_{\hat{ }CTGF-cgnt.12}.

TABLE 1

| | | Fraction dsRNA | | | Fraction dsRNA | | | Fraction dsRNA | | |
|---|---|---|---|---|---|---|---|---|---|---|
| | | released, 30 min | | | released, 90 min | | | released, 180 min | | |
| Hybrid Pair | | | | Effi- | | | Effi- | | | Effi- |
| Sense | Antisense | Non- | CTGF- | ciency | Non- | CTGF- | ciency | Non- | CTGF- | ciency |
| Hybrid | Hybrid | triggered | triggered | Score | triggered | triggered | Score | triggered | triggered | Score |
| sH_{DOWN·0bp} | aH_{UP·0bp} | 0.07 ± | 0.04 ± | 0.03 | 0.10 ± | 0.06 ± | 0.04 | 0.07 ± | 0.06 ± | 0.05 |
| | | 0.004 | 0.01 | | 0.03 | 0.01 | | 0.05 | 0.03 | |
| sH_{DOWN·+1bp} | aH_{UP·+1bp} | 0.09 ± | 0.08 ± | 0.07 | 0.08 ± | 0.11 ± | 0.15 | 0.08 ± | 0.12 ± | 0.20 |
| | | 0.02 | 0.02 | | 0.02 | 0.06 | | 0.08 | 0.04 | |
| sH_{DOWN·+2bp} | aH_{UP·+2bp} | 0.05 ± | 0.18 ± | 0.62 | 0.05 ± | 0.31 ± | 1.79 | 0.06 ± | 0.40 ± | 2.69 |
| | | 0.03 | 0.10 | | 0.01 | 0.16 | | 0.05 | 0.03 | |
| sH_{DOWN·+3bp} | aH_{UP·+3bp} | 0.08 ± | 0.39 ± | 2.01 | 0.06 ± | 0.49 ± | 4.00 | 0.06 ± | 0.63 ± | 6.29 |
| | | 0.01 | 0.13 | | 0.02 | 0.08 | | 0.02 | 0.01 | |
| sH_{DOWN·+4bp} | aH_{UP·+4bp} | 0.07 ± | 0.60 ± | 5.28 | 0.06 ± | 0.60 ± | 5.63 | 0.04 ± | 0.67 ± | 10.77 |
| | | 0.04 | 0.05 | | 0.01 | 0.06 | | 0.02 | 0.04 | |
| sH_{ΛCTGF12/8} | aH_{ΛCTGF-cgnt·12} | 0.05 ± | 0.66 ± | 9.5 | 0.06 ± | 0.78 ± | 9.7 | 0.07 ± | 0.85 ± | 10.0 |
| | | 0.02 | 0.06 | | 0.02 | 0.06 | | 0.03 | 0.04 | |
| sH_{ΛCTGF·12/12} | aH_{ΛCTGF-cgnt·12} | 0.05 ± | 0.66 ± | 8.1 | 0.09 ± | 0.81 ± | 7.6 | 0.11 ± | 0.85 ± | 6.8 |
| | | 0.02 | 0.05 | | 0.01 | 0.05 | | 0.05 | 0.04 | |
| sH_{ΛCTGF·16/8} | aH_{ΛCTGF-cgnt·12} | 0.04 ± | 0.69 ± | 13.3 | 0.05 ± | 0.78 ± | 12.0 | 0.07 ± | 0.83 ± | 10.5 |
| | | 0.01 | 0.09 | | 0.01 | 0.06 | | 0.02 | 0.04 | |
| sH_{ΛCTGF·20/8} | aH_{ΛCTGF-cgnt·12} | 0.02 ± | 0.66 ± | 17.8 | 0.04 ± | 0.83 ± | 17.5 | 0.05 ± | 0.79 ± | 13.6 |
| | | 0.01 | 0.06 | | 0.01 | 0.06 | | 0.02 | 0.05 | |
| sH_{ΛCTGF12/8} | aH_{ΛCTGF-cgnt·16} | 0.04 ± | 0.24 ± | 1.5 | 0.05 ± | 0.39 ± | 3.0 | 0.09 ± | 0.46 ± | 2.4 |
| | | 0.01 | 0.08 | | 0.02 | 0.04 | | 0.01 | 0.07 | |
| sH_{ΛCTGF·12/12} | aH_{ΛCTGF-cgnt·16} | 0.06 ± | 0.37 ± | 2.5 | 0.09 ± | 0.48 ± | 2.6 | 0.11 ± | 0.55 ± | 2.8 |
| | | 0.01 | 0.09 | | 0.04 | 0.02 | | 0.04 | 0.03 | |
| sH_{ΛCTGF·16/8} | aH_{ΛCTGF-cgnt·16} | 0.05 ± | 0.61 ± | 7.5 | 0.05 ± | 0.63 ± | 7.2 | 0.07 ± | 0.67 ± | 6.5 |
| | | 0.01 | 0.13 | | 0.01 | 0.02 | | 0.02 | 0.03 | |
| sH_{ΛCTGF·20/8} | aH_{ΛCTGF-cgnt·16} | 0.03 ± | 0.50 ± | 9.4 | 0.04 ± | 0.59 ± | 9.7 | 0.05 ± | 0.64 ± | 9.2 |
| | | 0.01 | 0.06 | | 0.004 | 0.05 | | 0.01 | 0.02 | |

TABLE 2

| +0 bp | | Non-Triggered | | | CTGF-Triggered | | |
|---|---|---|---|---|---|---|---|
| hybrid pair | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | * | ns | ns | — | | |
| | 90 min | ns | ns | ns | ns | — | |
| | 180 min | ns | ns | ns | ns | ns | — |
| +1 bp | | Non-Triggered | | | CTGF-Triggered | | |
| hybrid pair | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | ns | ns | ns | — | | |
| | 90 min | ns | ns | ns | ns | — | |
| | 180 min | ns | ns | ns | ns | ns | — |
| +2 bp | | Non-Triggered | | | CTGF-Triggered | | |
| hybrid pair | | 30 min | 90 min | 180 min | 30 min | 90 min | 180 min |
| Non- | 30 min | — | | | | | |
| Triggered | 90 min | ns | — | | | | |
| | 180 min | ns | ns | — | | | |
| CTGF- | 30 min | ns | ns | * | — | | |
| Triggered | 90 min | ns | ns | ns | ns | — | |
| | 180 min | ** | ** | * | ns | ns | — |
| +3 bp | | Non-Triggered | | | CTGF-Triggered | | |
| hybrid pair | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | ns | * | * | — | | |
| | 90 min | * | ** | * | ns | — | |
| | 180 min | *** | *** | *** | ns | ns | — |
| +4 bp | | Non-Triggered | | | CTGF-Triggered | | |
| hybrid pair | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | ** | ** | ** | — | | |
| | 90 min | ** | ** | ** | ns | — | |
| | 180 min | *** | ** | ** | ns | * | — |

TABLE 3
No trigger, 30 m

| | +0 bp | +1 bp | +2 bp | +3 bp | +4 bp |
|---|---|---|---|---|---|
| +0 bp | — | | | | |
| +1 bp | ns | — | | | |
| +2 bp | ns | ns | — | | |
| +3 bp | ns | ns | ns | — | |
| +4 bp | ns | ns | ns | ns | — |

CTGF trigger, 30 m

| | +0 bp | +1 bp | +2 bp | +3 bp | +4 bp |
|---|---|---|---|---|---|
| +0 bp | — | | | | |
| +1 bp | ns | — | | | |
| +2 bp | ns | ns | — | | |
| +3 bp | * | * | ** | — | |
| +4 bp | ** | ** | ** | * | — |

No trigger, 90 m

| | +0 bp | +1 bp | +2 bp | +3 bp | +4 bp |
|---|---|---|---|---|---|
| +0 bp | — | | | | |
| +1 bp | ns | — | | | |
| +2 bp | ns | ns | — | | |
| +3 bp | ns | ns | ns | — | |
| +4 bp | ns | ns | ns | ns | — |

CTGF trigger, 90 m

| | +0 bp | +1 bp | +2 bp | +3 bp | +4 bp |
|---|---|---|---|---|---|
| +0 bp | — | | | | |
| +1 bp | ns | — | | | |
| +2 bp | ns | ns | — | | |
| +3 bp | * | ** | ns | — | |
| +4 bp | ** | ** | * | * | — |

No trigger, 180 m

| | +0 bp | +1 bp | +2 bp | +3 bp | +4 bp |
|---|---|---|---|---|---|
| +0 bp | — | | | | |
| +1 bp | ns | — | | | |
| +2 bp | ns | ns | — | | |
| +3 bp | ns | ns | ns | — | |
| +4 bp | ns | ns | ns | * | — |

CTGF trigger, 180 m

| | +0 bp | +1 bp | +2 bp | +3 bp | +4 bp |
|---|---|---|---|---|---|
| +0 bp | — | | | | |
| +1 bp | * | — | | | |
| +2 bp | ** | * | — | | |
| +3 bp | *** | ** | ** | — | |
| +4 bp | ** | ** | ** | ns | — |

### Example 3

This example demonstrates that strand responsive structural element can act to conditionally induce strand exchange between RNA/DNA hybrids.

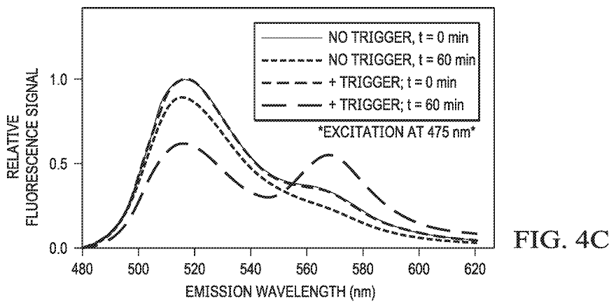

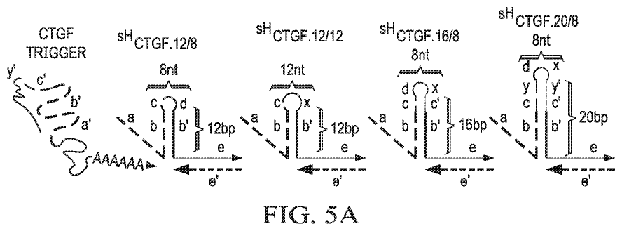

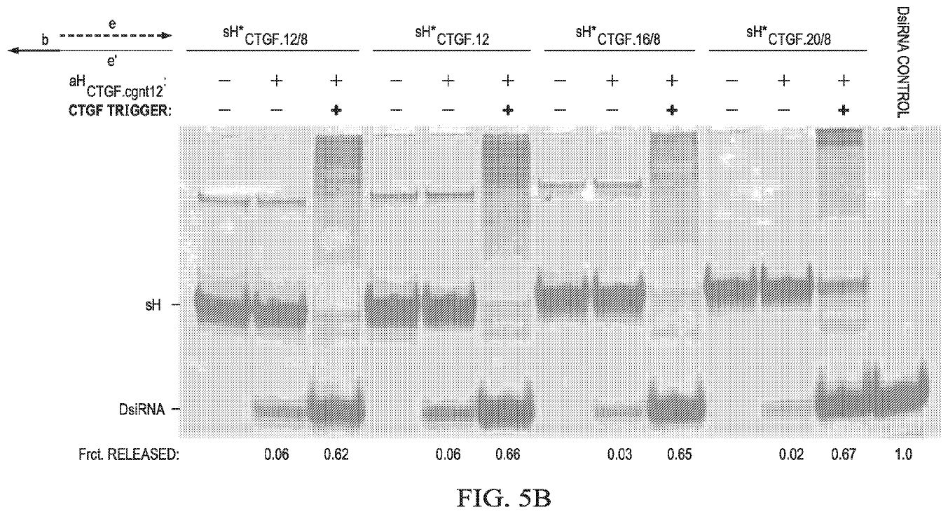

In an alternative approach for the implementation of conditional function within an RNA/DNA hybrid system, hybrid pairs were designed in which the accessibility of the toehold(s) needed to facilitate strand exchange was altered based on the presence or absence of a specific RNA target sequence. Although the adjacent targeting hybrid system described above performs its designed conditional function to release dsRNA, the fraction of dsRNA release for the best performing hybrid pair topped out at 0.67 after three hours. This second approach was pursued in an attempt to improve the efficiency of strand exchange and increase conditional dsRNA release. The “traditional” RNA/DNA hybrid methodology requiring the hybridization of complementary toeholds to one another for strand exchange serves as the basis of the conditional activation. The single stranded toeholds were designed as “exchange toeholds” because they are assist with strand exchange. To create a hybrid system responsive to conditional activation, a structured hairpin element was incorporated in the DNA strand immediately adjacent to the RNA/DNA hybrid duplex region of the sense hybrid (FIG. 4A). This DNA hairpin ultimately controls the reassembly fate of the split functional RNA. In its initial folded state, the DNA hairpin was designed to sequester the entire length of the exchange toehold sequence within its helical stem, preventing the toehold from readily interacting with the complementary exchange toehold of the cognate antisense hybrid. The resulting hybrid pair initially exists in an “off” state that was unable to initiate strand exchange.

A new single stranded toehold, termed the “trigger toehold,” was then implemented as a means to control the conditional activation of the hybrid by altering the accessibility of the exchange toehold imbedded within the DNA hairpin upon recognition of a specific RNA target sequence (FIG. 4A). This single-stranded trigger toehold within the sense hybrid was positioned at the 5′ end of the DNA strand adjacent to the DNA hairpin (at the side opposite the RNA/DNA hybrid region). By designing the sequence of the trigger toehold and the adjacent 5′ side of the DNA hairpin to be fully complementary to a region of an RNA target (e.g., mRNA), hybridization of the target to the trigger toehold unzips the adjacent DNA hairpin and exposes the exchange toehold. Once the exchange toehold has been liberated, the complementary exchange toeholds of the hybrid pair can facilitate a strand exchange event and release a dsRNA output (FIG. 4A). It was intended that this method of exchange toehold recognition, whereby the hybridization of complementary toeholds to one another forms a single duplex that can be directly extended by stacking additional DNA base pairs formed during RNA/DNA hybrid strand exchange, will exert a greater kinetic and/or thermodynamic drive than the three-way junction dependent method employed within the adjacent targeting system.

To illustrate the function of this “inducible” hybrid system, conditional hybrid constructs were designed to release a 25/27-mer DsiRNA when triggered by a fragment of the CTGF mRNA (target sequence). The DNA strand of the sense hybrid was designed to contain a central hairpin with a 12 base pair stem and 8 nucleotide loop. This sense hybrid was referred to as “sH_{\hat{ }CTGF.12/8}”, as the hybrid was designed to stimulate dsRNA release in the presence of CTGF (“\hat{ }CTGF”) and contains a DNA hairpin composed of a 12 base pair stem and 8 nucleotide loop (“12/8”). The exchange toehold within sH_{\hat{ }CTGF.12/8} was 12 nucleotides in length and was initially completely sequestered within the DNA hairpin stem. The cognate partner hybrid was composed of an RNA/DNA hybrid duplex containing the DsiRNA antisense strand, with a 12 nucleotide extension of the DNA strand at its 3′ end to encode the complementary exchange toehold. This hybrid was referred to as aH_{\hat{ }CTGF-cgnt.12} to reflect that it contains a 12 nucleotide exchange toehold (“12”) and was the cognate partner (“cgnt”) to the CTGF-triggered sH hybrid (“\hat{ }CTGF”).

Non-denaturing PAGE and total nucleic acid staining was used to examine interactions occurring between the cognate hybrids, as well as between the hybrids and the trigger RNA (FIG. 4B). While not quantitative, initial analysis using a nucleic acid stain allowed for surveillance of all molecular species and products. As expected, no changes to the hybrids' electrophoretic mobility was observed when incubated together at 37° C. in the absence of the trigger RNA, indicating that no interaction occurs between the hybrids and no dsRNA was released. Introduction of the RNA target activates sH_{\hat{ }CTGF.12/8} and induces the release of a dsRNA product when aH_{\hat{ }CTGF-cognt12} was also present. Higher migrating species are also observed when both hybrids are co-incubated with the trigger RNA. One of the high migrating bands corresponds to the expected waste product as indicated by similar migration of a control assembled from the RNA target and two DNA strands. An even slower migrating band was also observed and was likely to be a 5-molecule intermediate complex. FRET experiments were performed to further verify the generation of the expected double stranded RNA product in the presence of the trigger molecule (FIG. 4C). The cognate hybrids used for these FRET studies had a 3′ donor fluorophore on the RNA sense strand, and a 5′ acceptor fluorophore on the RNA antisense strand. In the absence of the RNA target, the FRET-labeled hybrid pair show no significant change in their emission spectrum after one hour at 37° C. However, one hour after the introduction of the CTGF trigger a large decrease in donor emission (˜515 nm) and increase in acceptor fluorescence (˜565 nm) was observed, indicating formation of the DsiRNA duplex product.

### Example 4

This example demonstrates that dsRNA release from cognate RNA/DNA hybrids can be optimized by alteration of structural elements.

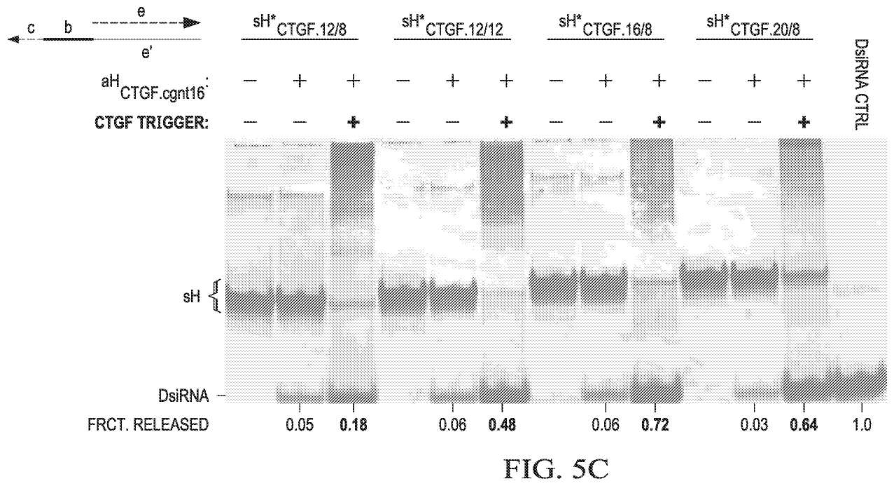

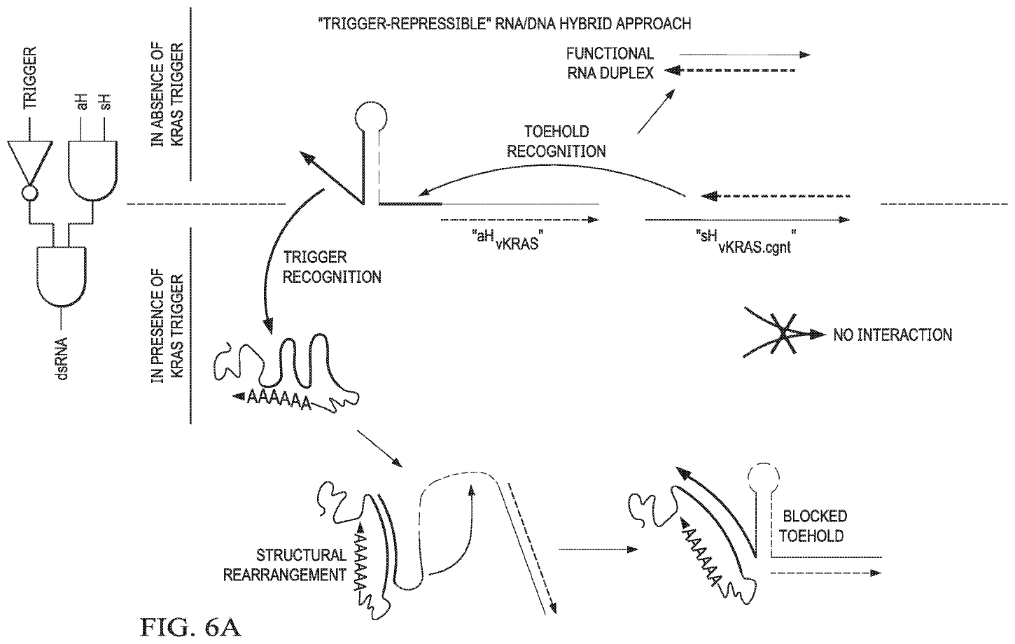

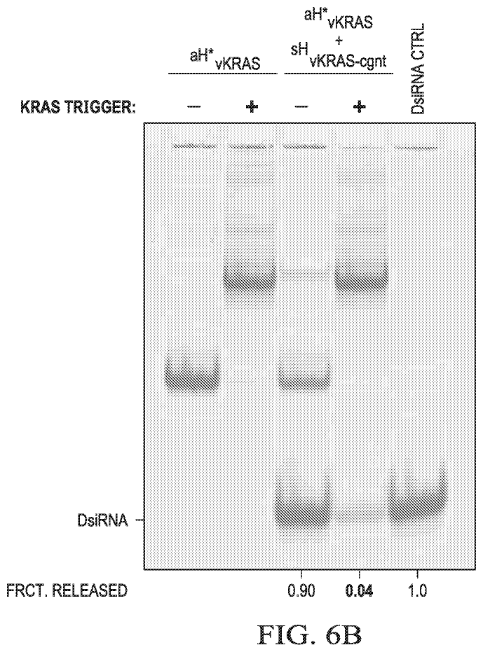

Within the inducible hybrid system, the accessibility of one exchange toehold was impeded by being sequestered within a responsive DNA hairpin. This toehold becomes liberated upon opening of the hairpin in the presence of an RNA target and allows for strand exchange to proceed. As such, altering the stability of this responsive hairpin structure, as well as the length and accessibility of the liberated exchange toehold once the hairpin was open, can modulate the degree of strand exchange between a cognate hybrid pair. The initially characterized sH_{\hat{ }CTGF.12/8} hybrid contained a 12 base pair DNA hairpin stem capped by an 8 nucleotide loop. Three additional CTGF-triggered sH hybrids were designed to investigate how changing the structure of the responsive DNA hairpin affects strand exchange and dsRNA release (FIG. 5A). The first of these variants maintains a 12 base pair DNA hairpin stem but expands the hairpin loop from 8 to 12 nucleotides. This hybrid was denoted sH_{\hat{ }CTGF.12/12}. The two additional sH variants maintain the original 8 nucleotide hairpin loop, but contain hairpin stems of 16 and 20 base pairs in length. These hybrids were named sH_{\hat{ }CTGF.16/8} and sH_{\hat{ }CTGF.20/8}, respectively.

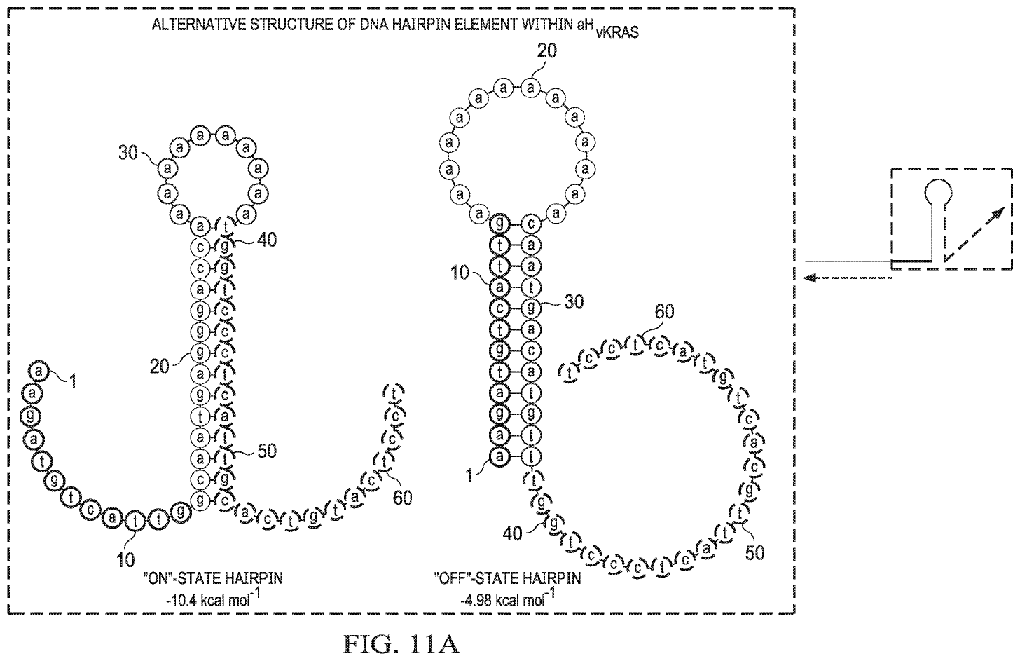

Each of the four sH_{\hat{ }CTGF} constructs were assembled with a fluorescently labeled RNA sense strand to quantitatively examine for their ability to liberate a dsRNA duplex following strand exchange with aH_{\hat{ }CTGF-cgnt.12} in the presence and absence of the CTGF trigger RNA (FIG. 5B). Interestingly, analysis using fluorescently labeled constructs revealed that the various sH_{\hat{ }CTGF}/aH_{\hat{ }CTGF-cgnt.12} hybrid pairs release a small fraction of dsRNA (i.e., “leaking”) when incubated together in absence of the trigger RNA. This was not originally observed in the initial qualitative experiments that utilized staining with ethidium bromide. The degree of non-triggered release among pairs of hybrid constructs was relatively minor after 30 minutes (2-5% of signal) and was observed to marginally increase over time for each variant sH_{\hat{ }CTGF} construct paired with aH_{\hat{ }CTGF-cgnt.12} (FIG. 5C). sH_{\hat{ }CTGF.20/8}, which was predicted to contain the most stable hairpin stem (FIG. 10), exhibited the smallest degree of non-triggered DsiRNA release compared to other hybrids pairs after 30 minutes. Likewise, sH_{\hat{ }CTGF.12/12} was predicted to have the weakest hairpin structure and displayed the greatest extent of non-triggered DsiRNA release after 30 minutes. This trend persists at longer time points, however, differences in non-triggered DsiRNA release among variant hybrids pairs were not all statistically significant, especially at longer time points (Table 4, statistical significance in the difference of dsRNA fraction released from differing inducible sH_{\hat{ }CTGF} hybrids paired with either aH_{\hat{ }CTGF-cgnt.12} or aH_{\hat{ }CTGF-cgnt.16} at a single time point, in presence or absence of the CTGF RNA trigger. P-values indicated as follows: not significant (ns) if >0.05; * if <0.05; ** if <0.01).

TABLE 4
CTGF Inducible sense hybrids after 30 minutes, no trigger

| | Paired with aH_{ΛCTGF-cgnt·12:} | | | | | Paired with aH_{ΛCTGF-cgnt·16:} | | | |
|---|---|---|---|---|---|---|---|---|---|
| | sH_{ΛCTGF·12/8} | sH_{ΛCTGF·12/12} | sH_{ΛCTGF·16/8} | sH_{ΛCTGF·20/8} | | sH_{ΛCTGF·12/8} | sH_{ΛCTGF·12/12} | sH_{ΛCTGF·16/8} | sH_{ΛCTGF·20/8} |
| sH_{ΛCTGF12/8} | — | | | | sH_{ΛCTGF12/8} | — | | | |
| sH_{ΛCTGF·12/12} | ns | — | | | sH_{ΛCTGF·12/12} | ns | — | | |
| sH_{ΛCTGF·16/8} | ns | ns | — | | sH_{ΛCTGF·16/8} | ns | ns | — | |
| sH_{ΛCTGF·20/8} | ns | * | * | — | sH_{ΛCTGF·20/8} | ns | ns | ns | — |

CTGF Inducible sense hybrids after 30 minutes, in presence of CTGF trigger

| | Paired with aH_{ΛCTGF-cgnt·12:} | | | | | Paired with aH_{ΛCTGF-cgnt·16:} | | | |
|---|---|---|---|---|---|---|---|---|---|
| | sH_{ΛCTGF·12/8} | sH_{ΛCTGF·12/12} | sH_{ΛCTGF·16/8} | sH_{ΛCTGF·20/8} | | sH_{ΛCTGF·12/8} | sH_{ΛCTGF·12/12} | sH_{ΛCTGF·16/8} | sH_{ΛCTGF·20/8} |
| sH_{ΛCTGF12/8} | — | | | | sH_{ΛCTGF12/8} | — | | | |
| sH_{ΛCTGF·12/12} | ns | — | | | sH_{ΛCTGF·12/12} | ns | — | | |
| sH_{ΛCTGF·16/8} | ns | ns | — | | sH_{ΛCTGF·16/8} | ns | * | — | |
| sH_{ΛCTGF·20/8} | ns | ns | ns | — | sH_{ΛCTGF·20/8} | ns | * | ns | — |

CTGF Inducible sense hybrids after 90 minutes, no trigger

| | Paired with aH_{ΛCTGF-cgnt·12:} | | | | | Paired with aH_{ΛCTGF-cgnt·16:} | | | |
|---|---|---|---|---|---|---|---|---|---|
| | sH_{ΛCTGF·12/8} | sH_{ΛCTGF·12/12} | sH_{ΛCTGF·16/8} | sH_{ΛCTGF·20/8} | | sH_{ΛCTGF·12/8} | sH_{ΛCTGF·12/12} | sH_{ΛCTGF·16/8} | sH_{ΛCTGF·20/8} |
| sH_{ΛCTGF12/8} | — | | | | sH_{ΛCTGF12/8} | — | | | |
| sH_{ΛCTGF·12/12} | ns | — | | | sH_{ΛCTGF·12/12} | ns | — | | |
| sH_{ΛCTGF·16/8} | ns | * | — | | sH_{ΛCTGF·16/8} | ns | ns | — | |
| sH_{ΛCTGF·20/8} | ns | ** | ns | — | sH_{ΛCTGF·20/8} | ns | ns | ** | — |

CTGF Inducible sense hybrids after 90 minutes, in presence of CTGF trigger

| | Paired with aH_{ΛCTGF-cgnt·12:} | | | | | Paired with aH_{ΛCTGF-cgnt·16:} | | | |
|---|---|---|---|---|---|---|---|---|---|
| | sH_{ΛCTGF·12/8} | sH_{ΛCTGF·12/12} | sH_{ΛCTGF·16/8} | sH_{ΛCTGF·20/8} | | sH_{ΛCTGF·12/8} | sH_{ΛCTGF·12/12} | sH_{ΛCTGF·16/8} | sH_{ΛCTGF·20/8} |
| sH_{ΛCTGF12/8} | — | | | | sH_{ΛCTGF12/8} | — | | | |
| sH_{ΛCTGF·12/12} | ns | — | | | sH_{ΛCTGF·12/12} | ns | — | | |
| sH_{ΛCTGF·16/8} | ns | ns | — | | sH_{ΛCTGF·16/8} | * | * | — | |
| sH_{ΛCTGF·20/8} | ns | ns | ns | — | sH_{ΛCTGF·20/8} | *** | ns | ns | — |

CTGF Inducible sense hybrids after 180 minutes, no trigger

| | Paired with aH_{ΛCTGF-cgnt·12:} | | | | | Paired with aH_{ΛCTGF-cgnt·16:} | | | |
|---|---|---|---|---|---|---|---|---|---|
| | sH_{ΛCTGF·12/8} | sH_{ΛCTGF·12/12} | sH_{ΛCTGF·16/8} | sH_{ΛCTGF·20/8} | | sH_{ΛCTGF·12/8} | sH_{ΛCTGF·12/12} | sH_{ΛCTGF·16/8} | sH_{ΛCTGF·20/8} |
| sH_{ΛCTGF12/8} | — | | | | sH_{ΛCTGF12/8} | — | | | |
| sH_{ΛCTGF·12/12} | ns | — | | | sH_{ΛCTGF·12/12} | ns | — | | |
| sH_{ΛCTGF·16/8} | ns | ns | — | | sH_{ΛCTGF·16/8} | * | ns | — | |
| sH_{ΛCTGF·20/8} | ns | ns | ns | — | sH_{ΛCTGF·20/8} | ** | ns | * | — |

CTGF Inducible sense hybrids after 180 minutes, in presence of CTGF trigger

| | Paired with aH_{ΛCTGF-cgnt·12:} | | | | | Paired with aH_{ΛCTGF-cgnt·16:} | | | |
|---|---|---|---|---|---|---|---|---|---|
| | sH_{ΛCTGF·12/8} | sH_{ΛCTGF·12/12} | sH_{ΛCTGF·16/8} | sH_{ΛCTGF·20/8} | | sH_{ΛCTGF·12/8} | sH_{ΛCTGF·12/12} | sH_{ΛCTGF·16/8} | sH_{ΛCTGF·20/8} |
| sH_{ΛCTGF12/8} | — | | | | sH_{ΛCTGF12/8} | — | | | |
| sH_{ΛCTGF·12/12} | ns | — | | | sH_{ΛCTGF·12/12} | ns | — | | |
| sH_{ΛCTGF·16/8} | ns | * | — | | sH_{ΛCTGF·16/8} | * | * | — | |
| sH_{ΛCTGF·20/8} | ns | ns | ns | — | sH_{ΛCTGF·20/8} | * | * | ns | — |

Structural changes to the responsive DNA hairpin of the sH_{\hat{ }CTGF} hybrids resulted in negligible differences in trigger-induced dsRNA release between the four sH_{\hat{ }CTGF}/aH_{\hat{ }CTGF-cgnt.12} pairs assayed (FIG. 5C). However, these constructs did show a 12-18% improvement in conditional dsRNA release over the best performing adjacent targeting hybrid pair after three hour incubations with the CTGF trigger (see Table 1). The lack of differences in triggered DsiRNA release among the variant sH_{\hat{ }CTGF} constructs was somewhat surprising based on the predicted change in free energy (ΔΔG) between the unbound and CTGF trigger-bound states for each hybrid's responsive DNA element (FIG. 10). However, it may be that the favorable change in free energy for each construct upon trigger binding was so great (ΔΔG<−25 kcal mol⁻¹ for each) that the comparatively small differences in ΔΔG between the various sH_{\hat{ }CTGF} hybrids becomes inconsequential. Alternatively, differences in the ΔΔG of trigger binding could be offset by differences in steric accessibility of the newly liberated exchange toehold once the DNA hairpin has opened. Increasing the loop size or length of the hairpin stem increases the distance between the exchange toehold and the region bound by the RNA target once hybridized (FIG. 10). This could in turn alter the accessibility of the liberated exchange toehold to the incoming cognate hybrid. The sH_{\hat{ }CTGF.12/8}/aH_{\hat{ }CTGF-cgnt.12} hybrid pair has the shortest nucleotide distance between the region bound by the trigger and its exchange toehold, and time course FRET experiments indicate the observed rate constant of dsRNA release was slower for this hybrid pairing than for any of the other three sH_{\hat{ }CTGF} hybrids paired with aH_{\hat{ }CTGF-cgnt.12}.

Extending the length of the exchange toehold was also explored as a means to boost triggered dsRNA release within the CTGF-inducible hybrid system. A variant aH_{\hat{ }CTGF-cgnt} hybrid was designed containing a 16 nt toehold and was termed aH_{\hat{ }CTGF-cgnt.16}. The toehold of aH_{\hat{ }CTGF-cgnt.16} was designed to encode the same 12 nucleotide sequence as the aH_{\hat{ }CTGF-cgnt.12} toehold, with four additional nucleotides appended to the toehold's distal end. These 4 additional nucleotides are complementary to corresponding regions within sH_{\hat{ }CTGF.16/8} and sH_{\hat{ }CTGF.20/8} and result in complete pairing of the 16 nucleotide exchange toeholds between these cognate hybrids. However, these 4 added nucleotides at the distal end of the aH_{\hat{ }CTGF-cgnt.16} toehold do not have complementary sequences in sH_{\hat{ }CTGF.12/8} and sH_{\hat{ }CTGF.12/12} (FIG. 5A), leaving the distal end of the aH_{\hat{ }CTGF-cgnt.16} toehold unpaired.

Increasing the toehold length of the cognate antisense hybrid from 12 to 16 nucleotides was observed to have a negative impact on DsiRNA release when paired with any of the sH_{\hat{ }CTGF} variants. The use of aH_{\hat{ }CTGF-cgnt.16} in place of aH_{\hat{ }CTGF-cgnt.12} had a negligible effect on the degree of non-triggered release, but presented a large significant impediment to CTGF-triggered release in nearly all instances (Table 5, statistical significance in the difference of dsRNA fraction released for a given sH_{\hat{ }CTGF} hybrid paired with aH_{\hat{ }CTGF-cgnt.16} compared to aH_{\hat{ }CTGF-cgnt.12}, in either the presence or absence of the CTGF RNA trigger. P-values indicated as follows: not significant (ns) if >0.05; * if <0.05; ** if <0.01). The extent of diminished triggered-release was most pronounced when aH_{\hat{ }CTGF-cgnt.16} was paired with sH_{\hat{ }CTGF.12/8} and sH_{\hat{ }CTGF.12/12}, suggesting that having non-complementary nucleotides at the distal end of the aH_{\hat{ }CTGF-cgnt.16} toehold interferes in some manner with the ability of the hybrids to promote the strand exchange reaction. As a way to compare the overall performance of the each conditionally-active hybrid pair an “efficiency score” was determined for each time point examined. This efficiency score metric was calculated as the product of the fraction of triggered dsRNA release and the signal-to-noise ratio (triggered/non-triggered release). Larger scores indicated greater efficiency of conditional dsRNA release. Out of the eight pairs of CTGF-inducible hybrids and the five sets of adjacent-targeting hybrids, the sH_{\hat{ }CTGF.20/8}/aH_{\hat{ }CTGF-cgnt.12} pairing displays the highest efficiency score for each time interval that was examined (see Table 1).

TABLE 5

| | | 30 min | 90 min | 180 min |
|---|---|---|---|---|
| Non-Triggered | sH_{ΛCTGF12/8} | ns | ns | ns |
| | sH_{ΛCTGF.12/12} | ns | ns | ns |
| | sH_{ΛCTGF.16/8} | ns | ns | ns |
| | sH_{ΛCTGF.20/8} | ns | ns | ns |
| CTGF-Triggered | sH_{ΛCTGF12/8} | * | ** | * |
| | sH_{ΛCTGF.12/12} | * | * | * |
| | sH_{ΛCTGF.16/8} | ns | * | * |
| | sH_{ΛCTGF.20/8} | * | * | ns |

### Example 5

This example demonstrates that redesigned responsive structural elements can be used to inhibit strand exchange and repress dsRNA release.

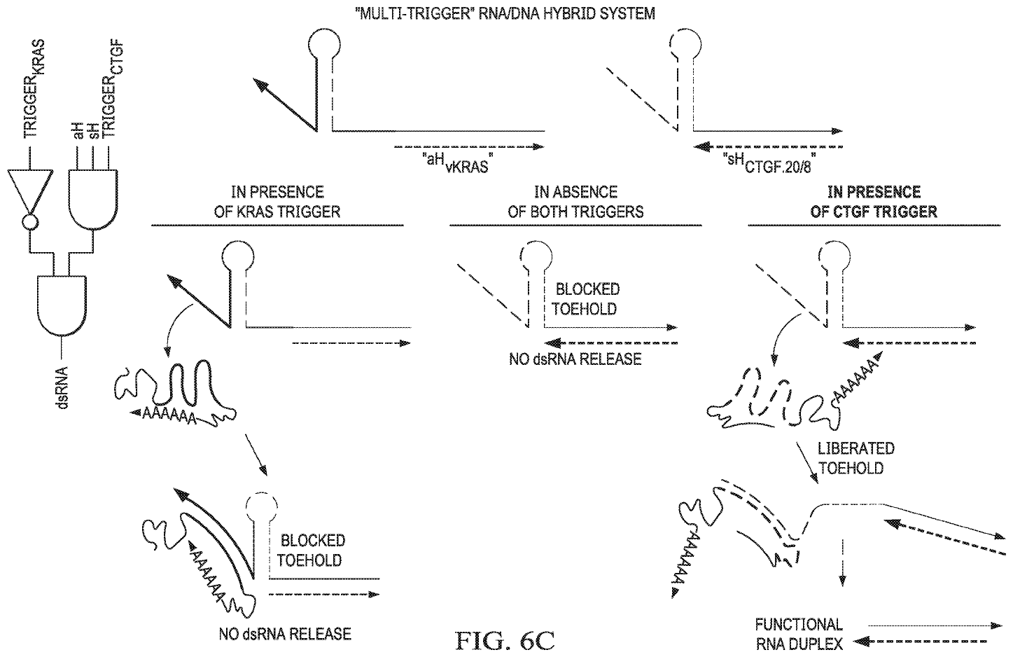

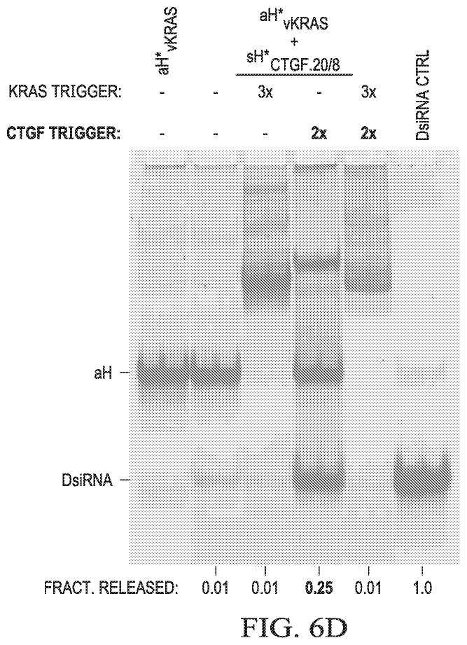

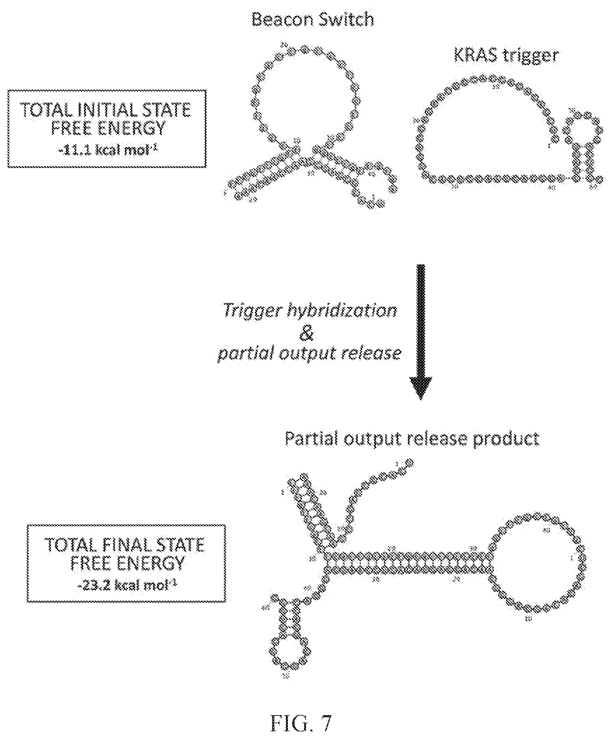

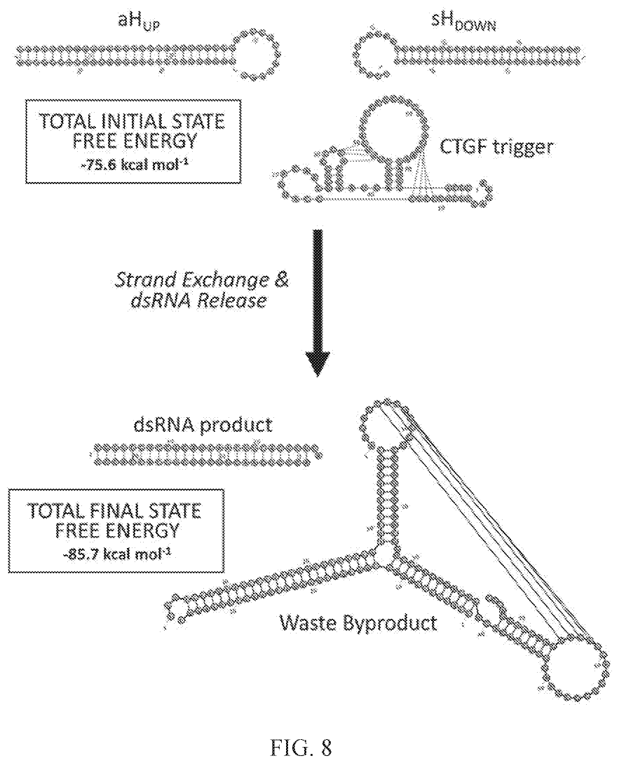

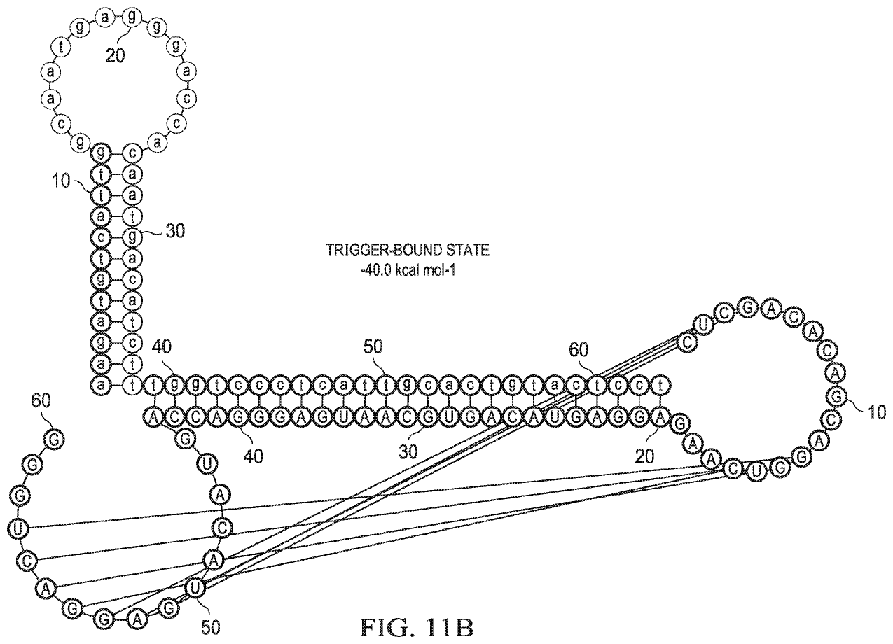

The concept and method of toehold sequestration used to impart conditional function within the trigger-inducible RNA/DNA hybrid system can modified and redesigned to instead allow for the repression of strand exchange in the presence of a specific RNA target and thereby expands the degree of control over dsRNA release. In this embodiment, both exchange toeholds are initially free to undergo strand exchange, but one becomes sequestered into a DNA hairpin when interaction with an RNA target facilitates a structural rearrangement of that hybrid's responsive structural element (FIG. 6A). Such a system would be of interest in situations where a cellular state of interest cannot be identified by the high expression of a particular RNA, but rather by a significant under expression of a specific RNA relative to the normal population.

Whereas the previously described inducible hybrid pairs contain a responsive hairpin element within the DNA strand of sH that was triggered by CTGF, the repressible hybrid pair contains a responsive DNA element within aH that was responsive to the KRAS mRNA-derived trigger. This new hybrid was termed “aH_{vKRAS}” to indicated that dsRNA release from the hybrid was negatively impacted by the KRAS trigger. In the absence of the cognate RNA target the most stable DNA fold of aH_{vKRAS} was that which results in a single stranded exchange toehold and a 14 base pair DNA hairpin (FIG. 11). When the trigger was present however, it can bind to the 3′ trigger toehold present in aH_{vKRAS} and proceed to unzip the 14 base pair hairpin, as the trigger was complementary to the entire 3′ side of the hairpin stem. As the initial 14 base pair hairpin can no longer form, a structural rearrangement can occur where the exchange toehold pairs to the 12 nucleotides that compose the apical loop of the original hairpin. This new hairpin structure makes the exchange toehold inaccessible to the cognate hybrid and represses the ability for the hybrid pair to release a dsRNA duplex (FIG. 6A).

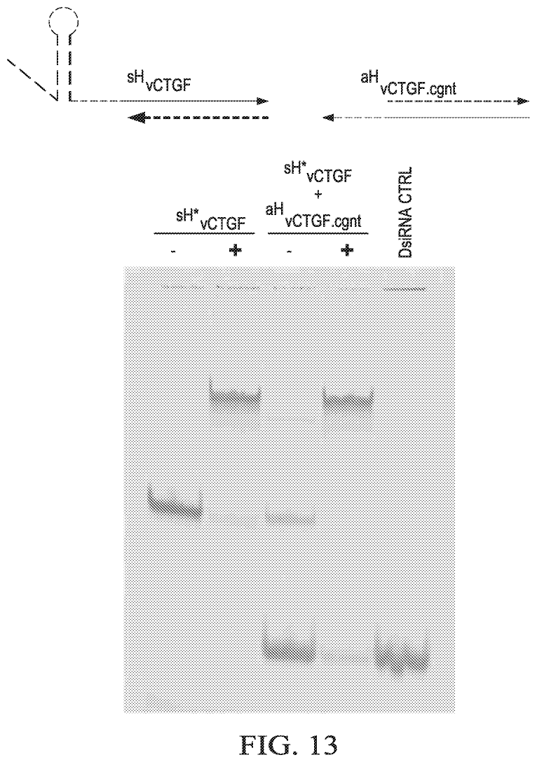

The ability to repress hybrid strand exchange was examined for aH_{vKRAS} with its cognate hybrid, “sH_{vKRAS-cgnt}”, that contains a complementary 12 nucleotide DNA exchange toehold extending from its RNA/DNA hybrid region. Analysis by non-denaturing PAGE at several time points illustrates that the cognate hybrids successfully undergo strand exchange and release dsRNA in the absence of the KRAS trigger (FIG. 6B). However, when the KRAS trigger and sH_{vKRAS-cgnt} hybrid are premixed and added simultaneously to aH_{vKRAS}, DsiRNA release was repressed more than 3-fold compared to in the absence of KRAS. A second context was also examined, where aH_{vKRAS} was permitted to interact with the KRAS trigger for five minutes prior to the addition of the cognate sH_{vKRAS-cgnt} hybrid. This scenario allowed the responsive DNA hairpin to rearrange and adopt its alternative “off”-state structure before the cognate exchange toehold was present in the reaction mix. In this context DsiRNA release was reduced 12-fold after 30 minutes at 37° C. compared to in the absence of the target, and maintains more than 7-fold repression after 3 hours. FRET experiments further illustrate that strand exchange occurs quickly and efficiently in the absence of the KRAS target, but was severely impeded upon introduction of KRAS (FIG. 13).

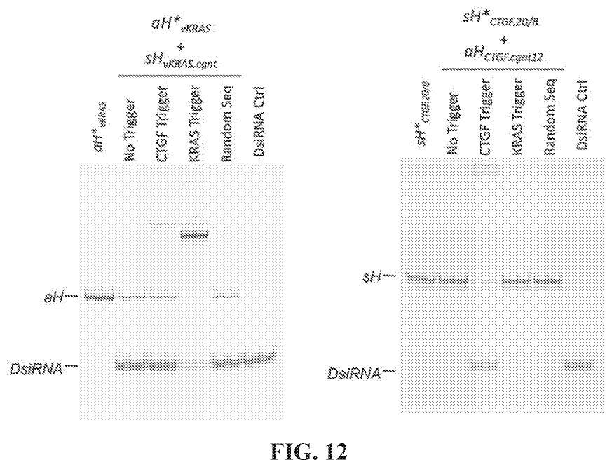

To illustrate that repression of dsRNA release was dependent on the presence of a trigger RNA with a specific nucleotide sequence, additional non-cognate trigger molecules were co-incubated with the repressive hybrid pair. Neither of the non-cognate trigger molecules tested resulted in a reduction in dsRNA release (FIG. 12). This same degree of trigger specificity was observed for the CTGF-inducible hybrid system, as the sH_{\hat{ }CTGF.20/8}/aH_{\hat{ }CTGF-cgnt.12} hybrid pair are only observed to initiate dsRNA release in the presence of the CTGF trigger, and not when co-incubated with non-cognate trigger molecules (FIG. 12). An orthogonal trigger-repressible system was also designed that was responsive to CTGF rather than KRAS, as a means to demonstrate versatility in accommodating various trigger sequence inputs, as well an ability to position the response element at different locations within this generalized conditional system. In this system, the CTGF responsive DNA element was added to the sense hybrid rather than the antisense hybrid. Nonetheless, this cognate hybrid pair (sH_{vCTGF}/aH_{vCTGF-cgnt}) displays a repressed ability to generate dsRNA in the presence of the CTGF target, as intended (FIG. 13).

### Example 6

This example demonstrates that cognate hybrid pairs with multiple responsive elements allow for multi-trigger regulation.

Because the strand change reaction between cognate hybrid partners was dependent on the accessibility of a specific toehold sequence (exchange toehold) present on each of the two hybrids, it was possible to generate a system in which the accessibility of each toehold was under the control of a different RNA target sequence. In the case of the trigger-repressible hybrids, such as aH_{vKRAS}, the trigger RNA imparts no sequence constraints on the exchange toehold and allows the exchange toehold to be any sequence that permits proper folding.

With this in mind, the exchange toehold of construct aH_{vKRAS} was designed to be complementary to the exchange toehold of the sH_{\hat{ }CTGF} hybrids characterized previously. Hybrid construct sH_{\hat{ }CTGF.20/8} was partnered with aH_{vKRAS} to generate a pair of conditional RNA/DNA hybrids whose function was dependent on the presence or absence of two RNA targets, CTGF and KRAS (FIG. 6C). The strand exchange reaction between these two hybrids was initially inhibited, as sH_{\hat{ }CTGF.20/8} initially exists in an “off” state and requires interaction with the CTGF trigger to promote strand exchange. aH_{vKRAS} was initially in an active state, however, the exchange toehold of aH_{vKRAS} becomes inaccessible upon interaction with the KRAS trigger. For efficient strand exchange to occur between this hybrid pair, the presence of the CTGF trigger was required, as well as the absence of the KRAS trigger.

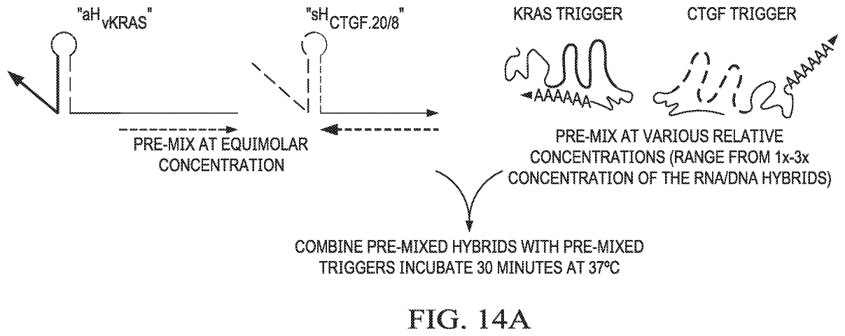

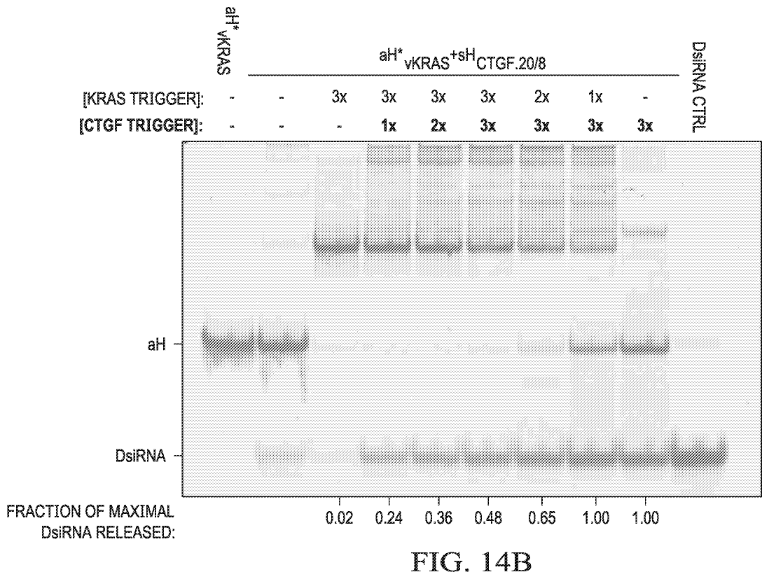

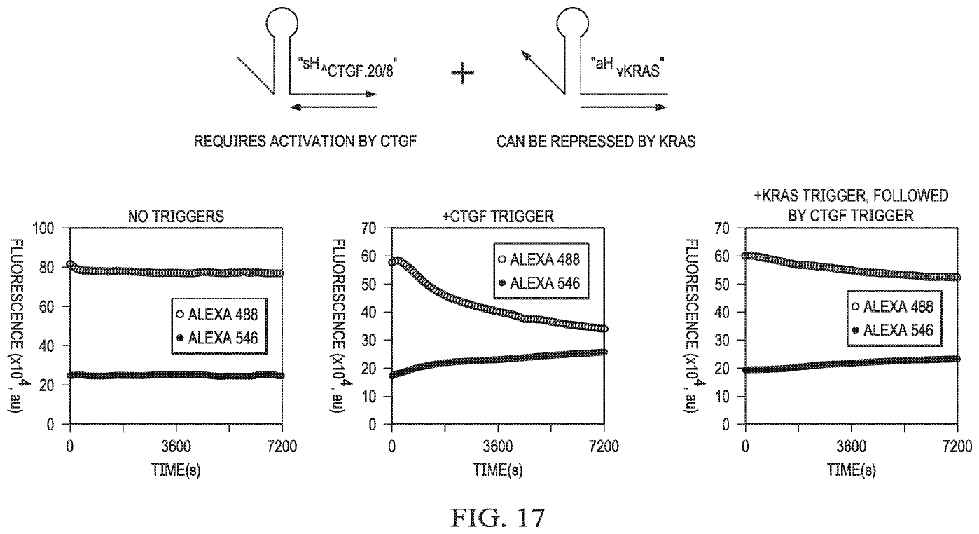

The degree to which dsRNA could be conditionally released from this cognate hybrid pair was assessed by non-denaturing PAGE (FIG. 6D) and FRET (FIG. 17). In the absence of any trigger molecules, the sH_{\hat{ }CTGF.20/8}/aH_{vKRAS} hybrid pair releases very small amounts of dsRNA when co-incubated. The addition of the KRAS trigger to the hybrid pair reduces the degree of dsRNA release close to zero. However, when CTGF target was added rather than the KRAS target, substantial release of dsRNA product occurs, as expected. Sequential addition of the KRAS target followed by the CTGF target to the hybrid pair results in very little dsRNA generation, suggesting that aH_{vKRAS} inactivation by the KRAS target occurs relatively quickly. Additional characterization was performed to examine how differences in the relative concentration of the two triggers affect dsRNA release. Various ratios of CTGF and KRAS target molecules were premixed and added to the co-incubating sH_{\hat{ }CTGF.20/8}/aH_{vKRAS} hybrid pair. As might be expected, increasing the relative amount of CTGF target (activating) to KRAS target (deactivating) increases the extent of dsRNA release (FIGS. 14A and 14B). When equal amounts of the KRAS and CTGF targets are added to the hybrid pair, the degree of dsRNA release was about 60% of the maximum amount of dsRNA released when an excess of CTGF target was added to the hybrids in the absence of the KRAS target. However, when the ratio of CTGF/KRAS targets was varied away from 1:1, induction/repression of dsRNA release disproportionately favors the target that was present in greater amount, beyond what would be predicted based on the target stoichiometry (i.e.: when a 3:2 ratio of KRAS/CTGF was present, the fraction of dsRNA was less than 40% of the maximal dsRNA released in absence of any KRAS). See Tables 6, statistical significance in the difference of dsRNA fraction released from individual CTGF-inducible hybrid pairs at various time points, in presence or absence of the CTGF RNA trigger. P-values indicated as follows: not significant (ns) if >0.05; * if <0.05; ** if <0.01; *** if <0.001, and Table 7, statistical significance in the difference of dsRNA fraction released the KRAS-repressible hybrid pair aH_{vKRAS} and sH_{vKRAS-cgnt} in various molecular environments, at multiple time points. P-values indicated as follows: not significant (ns) if >0.05; * if <0.05; ** if <0.01; *** if <0.001.

### Example 7

This example demonstrates that three strand RNA/DNA hybrid constructs allow “activated” hybrids to dissociate from their cognate target sequence.

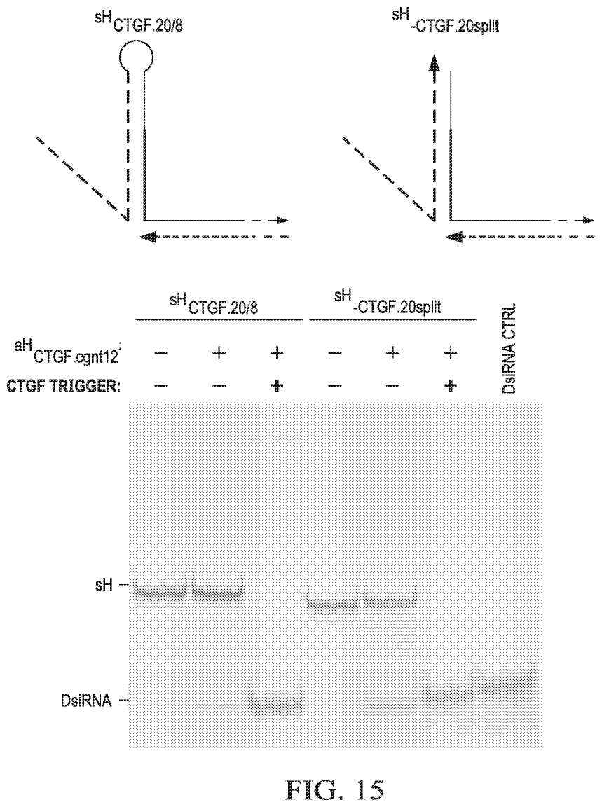

With both the inducible and repressible conditional systems described above, the entirety of the hybrid construct containing the trigger toehold remains bound to the RNA target molecule following recognition and hybridization of the trigger toehold. However, there are instances where the function of the conditional hybrid systems benefit from allowing their RNA/DNA hybrid domains to freely diffuse away from their cognate trigger following hybridization through their trigger toehold/domain. A three strand design approach was used to create an inducible hybrid that separates from the RNA target after hybridization. The design was based on that of the sH_{\hat{ }CTGF.20/8} hybrid. The 8 nucleotide hairpin loop was removed, splitting the 20 base pair hairpin into a duplex that assembles from two distinct DNA strands. One DNA strand retained the 5′ trigger toehold, while the other maintained the RNA/DNA hybrid region (FIG. 15). This new three-strand hybrid was called “sH_{\hat{ }CTGF.20split}” and works in conjunction with aH_{\hat{ }CTGF-cgnt.12}. Analysis by non-denaturing PAGE illustrates that the three-piece hybrid sH_{\hat{ }CTGF.20split} appears to function very similarly to that of sH_{\hat{ }CTGF 20/8}, although the three-piece hybrid has a slight increase in its degree of non-triggered dsRNA release (FIG. 15).

A similar approach was used to investigate a three-strand repressible hybrid construct based on the aH_{vKRAS} hybrid. A nick was positioned within the 5′ strand of the DNA hairpin, aiming to maintain stable formation of the initial 14 base pair hairpin and allow strand exchange in the absence of the KRAS trigger. Four different variants were designed to identify a nick position that retained the greatest conditional function. The function of the four variants partnered with sH_{vKRAS-cgnt} was examined by non-denaturing PAGE (FIG. 15). Each of the three-strand repressible systems tested show a diminished ability to promote desirable dsRNA release in the absence of the KRAS trigger compared to the original design. This may stem from the possibility that a larger fraction of the three-strand hybrids initially adopt their “off” state when assembled. However, some three-strand systems did retain their repressible function. “aH_{vKRAS.nick14}”, where the nick was placed immediately below the hairpin loop and preserving the entire 14 base pair stem, displayed the greatest degree of conditional function. Progressively moving the nick down the stem resulted in continued loss of the responsive function to the KRAS target.

TABLE 6

| sH_{ΛCTGF·12/8}/ | | Non-Triggered | | | CTGF-Triggered | | |
|---|---|---|---|---|---|---|---|
| aH_{ΛCTGF-cgnt·12} | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | ** | ** | ** | — | | |
| | 90 min | ** | *** | ** | ns | — | |
| | 180 min | *** | *** | ** | * | * | — |
| sH_{ΛCTGF·12/12}/ | | Non-Triggered | | | CTGF-Triggered | | |
| aH_{ΛCTGF-cgnt·12} | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | *** | ** | * | — | | |
| | 90 min | ** | *** | ** | ns | — | |
| | 180 min | *** | *** | ** | ** | ns | — |
| sH_{ΛCTGF·16/8}/ | | Non-Triggered | | | CTGF-Triggered | | |
| aH_{ΛCTGF-cgnt·12} | | 30 min | 90 min | 180 min | 30 min | 90 min | 180 min |
| Non-Triggered | 30 min | — | | | | | |
| | 90 min | * | — | | | | |
| | 180 min | ns | ns | — | | | |
| CTGF-Triggered | 30 min | ** | ** | * | — | | |
| | 90 min | ** | ** | ** | ns | — | |
| | 180 min | *** | *** | ** | ns | ns | — |
| sH_{ΛCTGF·20/8}/ | | Non-Triggered | | | CTGF-Triggered | | |
| aH_{ΛCTGF-cgnt·12} | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | ** | ** | ** | — | | |
| | 90 min | ** | ** | ** | ns | — | |
| | 180 min | ** | ** | *** | ns | ns | — |
| sH_{ΛCTGF·12/8}/ | | Non-Triggered | | | CTGF-Triggered | | |
| aH_{ΛCTGF-cgnt·16} | | 30 min | 90 min | 180 min | 30 min | 90 min | 180 min |
| Non-Triggered | 30 min | — | | | | | |
| | 90 min | ns | — | | | | |
| | 180 min | * | ns | — | | | |
| CTGF-Triggered | 30 min | * | ns | ns | — | | |
| | 90 min | ** | ** | ** | ns | — | |
| | 180 min | ** | * | * | ns | ns | — |
| sH_{ΛCTGF·12/12}/ | | Non-Triggered | | | CTGF-Triggered | | |
| aH_{ΛCTGF-cgnt·16} | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | * | * | ns | — | | |
| | 90 min | *** | *** | ** | ns | — | |
| | 180 min | *** | ** | ** | * | * | — |
| sH_{ΛCTGF·16/8}/ | | Non-Triggered | | | CTGF-Triggered | | |
| aH_{ΛCTGF-cgnt·16} | | 30 min | 90 min | 180 min | 30 min | 90 min | 180 min |
| Non-Triggered | 30 min | — | | | | | |
| | 90 min | ns | — | | | | |
| | 180 min | ns | ns | — | | | |
| CTGF-Triggered | 30 min | * | * | * | — | | |
| | 90 min | *** | *** | ** | ns | — | |
| | 180 min | *** | *** | ** | ns | ns | — |
| sH_{ΛCTGF·20/8}/ | | Non-Triggered | | | CTGF-Triggered | | |
| aH_{ΛCTGF-cgnt·16} | | 30 min | 90 min | 180 min | 30 min | 90 min | 180 min |
| Non-Triggered | 30 min | — | | | | | |
| | 90 min | ns | — | | | | |
| | 180 min | * | ns | — | | | |
| CTGF-Triggered | 30 min | ** | ** | ** | — | | |
| | 90 min | ** | ** | ** | ns | — | |
| | 180 min | *** | *** | *** | * | ns | — |

TABLE 7

| | | 30 minutes | | | 90 minutes | | | 180 minutes | | |
|---|---|---|---|---|---|---|---|---|---|---|
| | | | | aH_{vKRAS} + | | | aH_{vKRAS} + | | | aH_{vKRAS} + |
| | | | aH_{vKRAS} + | KRAS, | | aH_{vKRAS} + | KRAS, | | aH_{vKRAS} + | KRAS, |
| | | | mixture of | followed | | mixture | followed | | mixture | followed |
| | | aH_{vKRAS} + | KRAS/ | by | aH_{vKRAS} + | of KRAS/ | by | aH_{vKRAS} + | of KRAS/ | by |
| | | sH_{vKRAS-cgnt} | sH_{vKRAS-cgnt} | sH_{vKRAS-cgnt} | sH_{vKRAS-cgnt} | sH_{vKRAS-cgnt} | sH_{vKRAS-cgnt} | sH_{vKRAS-cgnt} | sH_{vKRAS-cgnt} | sH_{vKRAS-cgnt} |
| 30 | aH_{vKRAS} + | — | | | | | | | | |
| mi- | sH_{vKRAS-cgnt} | | | | | | | | | |
| nutes | aH_{vKRAS} + | ** | — | | | | | | | |
| | mixture of | | | | | | | | | |
| | KRAS/ | | | | | | | | | |
| | sH_{vKRAS-cgnt} | | | | | | | | | |
| | aH_{vKRAS} + | ** | ** | — | | | | | | |
| | KRAS, | | | | | | | | | |
| | followed | | | | | | | | | |
| | by | | | | | | | | | |
| | sH_{vKRAS-cgnt} | | | | | | | | | |
| 90 | aH_{vKRAS} + | * | *** | *** | — | | | | | |
| mi- | sH_{vKRAS-cgnt} | | | | | | | | | |
| nutes | aH_{vKRAS} + | ** | * | ** | *** | — | | | | |
| | mixture of | | | | | | | | | |
| | KRAS/ | | | | | | | | | |
| | sH_{vKRAS-cgnt} | | | | | | | | | |
| | aH_{vKRAS} + | ** | ** | * | *** | ** | — | | | |
| | KRAS, | | | | | | | | | |
| | followed | | | | | | | | | |
| | by | | | | | | | | | |
| | sH_{vKRAS-cgnt} | | | | | | | | | |
| 180 | aH_{vKRAS} + | ns | *** | *** | ns | *** | *** | — | | |
| mi- | sH_{vKRAS-cgnt} | | | | | | | | | |
| nutes | aH_{vKRAS} + | ** | ns | ** | *** | ns | ** | *** | — | |
| | mixture of | | | | | | | | | |
| | KRAS/ | | | | | | | | | |
| | sH_{vKRAS-cgnt} | | | | | | | | | |
| | aH_{vKRAS} + | *** | *** | * | *** | ** | * | *** | ** | — |
| | KRAS, | | | | | | | | | |
| | followed | | | | | | | | | |
| | by | | | | | | | | | |
| | sH_{vKRAS-cgnt} | | | | | | | | | |

### Example 8

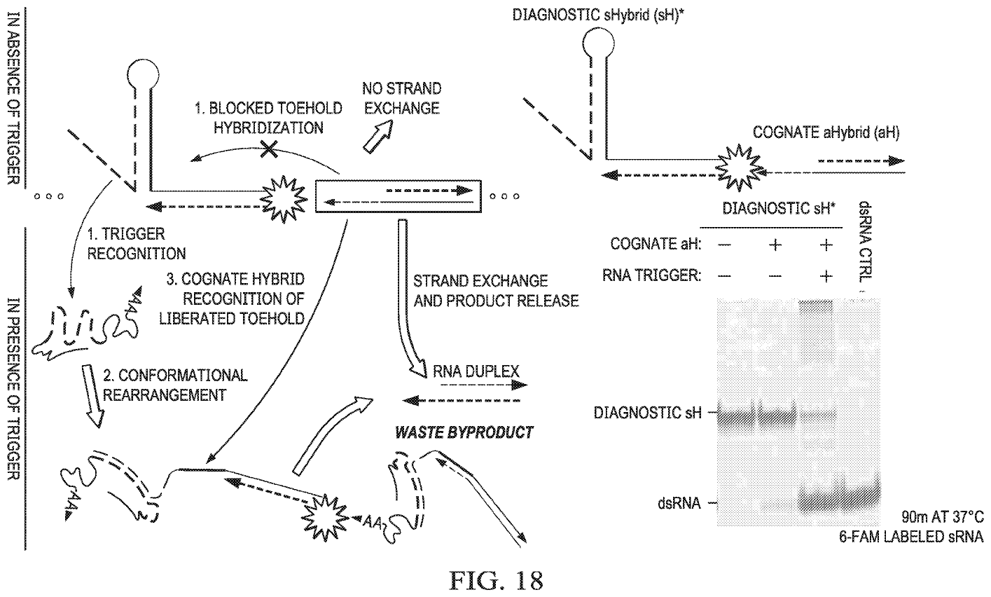

Initial characterizations were performed at 500 nM concentration of the inducible hybrid system, with 2-fold excess of trigger molecule (1 uM), in buffer, results as seen in FIG. 18. In order to assess the sensitivity of embodiments of the inducible hybrid system for diagnostic or therapeutic applications: (1) decreased concentration of the hybrid constructs and trigger were examined and (2) the reaction environment in which the system functions was further studied.

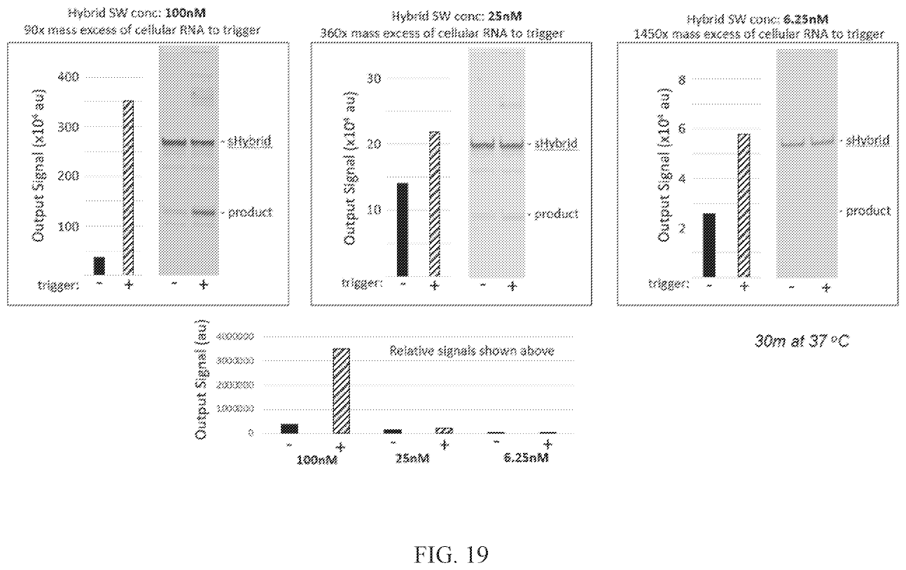

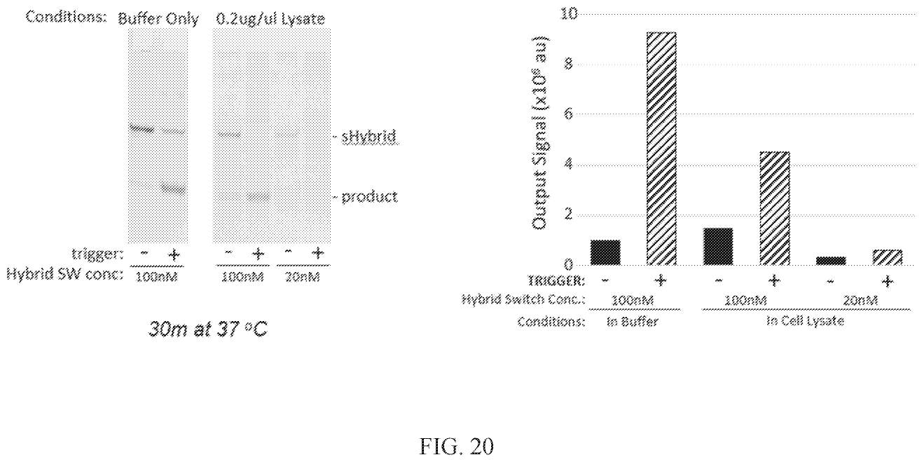

Lowering the concentration of the hybrids/trigger is important for both diagnostic and therapeutic applications. For purely diagnostic purposes, lowering the concentration of the system would lower the amount of cellular RNA that needs to be extracted and pooled from cells. For cell applications, high copy number mRNAs is present at sub/low nanomolar concentrations, which also appears to be the needed concentration of exogenous siRNAs for effective gene silencing. Each of the experiments presented in FIGS. 19 and 20 were performed at significantly reduced concentrations, compared to the experiments in buffer as seen in FIG. 18, and still show their intended function of releasing product in the presence of trigger (even at low nanomolar concentrations as in FIG. 19).

For experiments done in the presence of total cellular RNA (FIG. 19): Cellular RNA was extracted from cells using a commercially available kit. Fresh stocks of each individual hybrid construct and the RNA trigger fragment were assembled/folded the day of the experiment. The RNA trigger used was a fragment of the endogenous CTGF mRNA, 81nts (underlined nucleotides at 5′end are not part of native sequence) sequence: 5′gggaAAGACCUGUGCCUGCCAUUACAACUGUCCCGGAGACAAUGACAUCUUU GAAUCGCUGUACUACAGGAAGAUGUACGG 3′ (SEQ ID NO:62). Reactions were prepared by combining water, buffer and cellular RNA, to which hybrids and the RNA trigger were added. Reactions were allowed to incubate for a defined duration. In these particular experiments, the amount of output was determined by non-denaturing PAGE analysis.

For the purpose of being used purely as a diagnostic, the conditional hybrid system must discern one specific trigger RNA sequence from a pool of all RNAs that are present in the cell. In order to examine the sensitivity of the inducible hybrid system disclosed herein, experiments were conducted in reactions vessels containing total cellular RNA extracted from cultured cells. At the lowest concentration of hybrids/trigger tested (6.25 nM/12.5 nM, respectively), the mass of cellular RNA in the reaction vessel was greater than 1400-times that of the doped-in trigger fragment. The system still produced a detectable increase in the output signal under these conditions when the trigger was present vs when it was absent. This indicates that the different toeholds are able to find and discriminate their cognate partners despite the complex environment of heterogeneous RNAs.

The present inducible hybrid system is designed to be utilizable for in-cell functions, such as conditional therapeutics or in-cell diagnostics. The cellular environment is extremely complex compared to a test-tube environment. Toward in-cell application, the system was tested to see if it was functional in a slightly more controlled environment. To this end, experiments were conducted in cell lysate (which should contain nearly all molecular components of cells such as proteins, RNAs and small metabolites). To more closely mimic a cellular environment, this experiment was performed without the addition of nuclease inhibitors, as the inducible system might be considered to be susceptible to degradation by these nuclease proteins that exist in cells (FIG. 20). Even in this complex environment, the inducible hybrid system showed detectable conditional function at the lowest concentration assayed. This suggests that the inducible hybrid system should function in the cellular environment, as it functions in a test tube in the presence of cellular components.

For the experimental results shown in FIG. 20, cells were lysed with a lysis buffer that can be purchased or prepared in the lab. Fresh stocks of each individual hybrid construct and the RNA trigger fragment were assembled/folded the day of the experiment. The same CTGF trigger fragment was used as is given above with respect to the experimental methods for the results of FIG. 19. Reactions were prepared by combining water, buffer and cell lysate, to which hybrids and the RNA trigger were added. Reactions were allowed to incubate for a defined duration. The amount of output was determined by non-denaturing PAGE analysis.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. An DNA/RNA hybrid nucleic acid nanoparticle comprising:
(a) at least one trigger toehold or at least one exchange toehold, wherein each at least one trigger toehold and the at least one exchange toehold independently comprise DNA and/or RNA; and
(b) at least one single stranded RNA output strand,
wherein no portion of the at least one trigger toehold hybridizes to any portion of the at least one output strand, the at least one trigger toehold is complementary and hybridizes to a first target sequence when the nanoparticle is in the presence of the first target sequence, and the nanoparticle does not contain the target sequence, further wherein the nanoparticle comprises a sense construct and an antisense construct, wherein the sense construct and the antisense construct are not connected to each other, wherein the at least one output strand separates from the nanoparticle when the at least one trigger toehold hybridizes to the first target sequence.

2. The nanoparticle of claim 1, wherein the first target sequence is part of a mRNA.

3. (canceled)

4. The nanoparticle of claim 1, wherein the at least one output strand does not comprise 2′ modified nucleotides.

5. The nanoparticle of claim 1, wherein the at least one trigger toehold forms a loop that does not contain the at least one output strand and the nanoparticle comprises at least one strand that is complementary to the at least one output strand.

6. The nanoparticle of claim 1, wherein the sense construct comprises a first trigger toehold and a first output strand, the antisense construct comprises a second trigger toehold and a second output strand, and the first trigger toehold and the second trigger toehold are complementary to adjacent positions within the first target sequence.

7. The nanoparticle of claim 6, wherein when the first trigger toehold and the second trigger toehold hybridize to adjacent positions within the first target sequence, the first output strand hybridizes to the second output strand and forms a double stranded output strand that separates from the nanoparticle.

8. The nanoparticle of claim 6, wherein the sense construct comprises a first DNA strand comprising a sequence that is complementary to the first output strand and the antisense construct comprises a second DNA strand comprising a sequence that is complementary to the second output strand, wherein the first DNA strand is connected to the first trigger toehold and the second DNA strand is connected to the second trigger toehold.

9. The nanoparticle of claim 8, wherein the first DNA strand of the sense construct comprises from about 1 to about 10 nucleic bases between the first trigger toehold and the sequence that is complementary to the first output strand and the second DNA strand of the antisense construct comprises from about 1 to about 10 nucleic bases between the second trigger toehold and the sequence that is complementary to the second output strand, and the from about 1 to about 10 bases of the first and second DNA strands are complementary to each other.

10. The nanoparticle of claim 1, wherein the sense construct comprises at least one hairpin loop comprising a helical stem and a loop.

11. The nanoparticle of claim 10, wherein
(a) the sense construct comprises a first trigger toehold, a first exchange toehold, a first output strand, and a first DNA strand comprising a sequence that is complementary to the first output strand, and
(b) the antisense construct comprises a second output strand and a second exchange toehold that is connected to a second DNA strand comprising a sequence that is complementary to the second output strand.

12. The nanoparticle of claim 11, wherein the first exchange toehold is within the helical stem of the hairpin loop of the sense construct.

13. The nanoparticle of claim 11, wherein when the at least one trigger toehold hybridizes to the first target sequence, the hairpin loop is disrupted exposing the first exchange toehold such that the first exchange toehold can bind to the second exchange toehold allowing the first output strand to hybridize to the second output strand and thereby release the double stranded RNA output strand.

14. The nanoparticle of claim 11, wherein when the first target sequence is not in proximity to the sense construct, the hairpin loop is not disrupted and the first exchange toehold is kept within the helical stem of the hairpin loop, and a double stranded RNA output strand is not created by the first output strand hybridizing to the second output strand and a double stranded RNA output strand is not released by the nanoparticle.

15. The nanoparticle of claim 11, wherein the helical stem of the hairpin loop comprises from about 12 to about 50 base pairs and the loop of the hairpin loop comprises from about 8 to about 20 nucleotides.

16. The nanoparticle of claim 11, wherein the helical stem of the hairpin loop comprises from about 12 to about 20 base pairs and the loop of the hairpin loop comprises from about 8 to about 12 nucleotides.

17. The nanoparticle of claim 11, wherein the first exchange toehold is not within the helical stem of the hairpin loop.

18. The nanoparticle of claim 17, wherein when the at least one trigger toehold hybridizes to the first target sequence, the hairpin loop is disrupted sequestering the first exchange toehold, a double stranded RNA output strand is not created by the first output strand hybridizing to the second output strand and a double stranded RNA output strand is not released by the nanoparticle.

19. The nanoparticle of claim 11, wherein
(a) the sense construct further comprises a first helical loop with a first helical stem and a first hairpin loop and the first exchange toehold is sequestered within the first helical stem, and
(b) the antisense construct further comprises a second helical loop with a second helical stem and a second hairpin loop and the second exchange toehold is not within the second helical loop,
wherein the first exchange toehold is no longer sequestered within the first helical stem when the sense construct is hybridized to the first target sequence, wherein the second toehold becomes sequestered within the second helical loop when the antisense construct hybridizes to a second target sequence, the ratio of the amount of the first target sequence to the amount of the second target sequence in proximity to the sense construct and antisense construct that is sufficient to result in hybridization of the first trigger toehold to the first target sequence or the second trigger toehold to the second target sequence impacts the binding kinetics between the first exchange toehold and the first target sequence and the second toehold and the second target sequence, and the ratio of the amount of the first target sequence to the amount of the second target sequence is from about 1:1,000 to about 1,000:1.

20. The nanoparticle of claim 19, wherein the ratio of the amount of the first target sequence to the amount of the second target sequence is from about 1:100 to about 100:1.

21. The nanoparticle of claim 19, wherein the ratio of the amount of the first target sequence to the amount of the second target sequence is from about 1:3 to about 3:1.

22. The nanoparticle of claim 1, wherein the first target sequence comprises a nucleotide sequence encoding KRAS (SEQ ID NO: 61).

23. The nanoparticle of claim 1, wherein the first target sequence comprises a nucleotide sequence encoding CTGF (SEQ ID NO: 59).

24. The nanoparticle of claim 19, wherein the second target sequence comprises a nucleotide sequence encoding KRAS (SEQ ID NO: 61).

25. The nanoparticle of claim 19, wherein the second target comprises a nucleotide sequence encoding CTGF (SEQ ID NO: 59).

26. A composition comprising the nanoparticle of claim 1 and a pharmaceutically acceptable carrier.

27. A method of treating a patient with a disease or condition, the method comprising administering the nanoparticle of claim 1 to the patient.

28. A method of diagnosing a patient with a disease or condition, the method comprising
(a) administering the nanoparticle of claim 1 to the patient;
(b) observing the level of separated output strands in a patient sample and comparing the level of separated output strands to a threshold.

29. (canceled)


SEQUENCE LISTING (62 sequences)

SEQ ID NO: 1 - DNA, 43 bp, Artificial Sequence
  synthetic
tttgttcgtttcattgcactgtactcctcttggctcgctgtga

SEQ ID NO: 2 - RNA, 22 nt, Artificial Sequence
  synthetic
ucacgcgagccgaacgaacaaa

SEQ ID NO: 3 - RNA, 27 nt, Artificial Sequence
  synthetic
cgguggugcagaugaacuucaggguca

SEQ ID NO: 4 - DNA, 39 bp, Artificial Sequence
  synthetic
tgaccctgaagttcatctgcaccaccgagttgtaatggc

SEQ ID NO: 5 - RNA, 25 nt, Artificial Sequence
  synthetic
acccugaaguucaucugcaccaccg

SEQ ID NO: 6 - DNA, 37 bp, Artificial Sequence
  synthetic
ttgtctccgggacggtggtgcagatgaacttcagggt

SEQ ID NO: 7 - RNA, 27 nt, Artificial Sequence
  synthetic
cgguggugcagaugaacuucaggguca

SEQ ID NO: 8 - DNA, 40 bp, Artificial Sequence
  synthetic
tgaccctgaagttcatctgcaccaccggagttgtaatggc

SEQ ID NO: 9 - RNA, 25 nt, Artificial Sequence
  synthetic
acccugaaguucaucugcaccaccg

SEQ ID NO: 10 - DNA, 38 bp, Artificial Sequence
  synthetic
ttgtctccgggaccggtggtgcagatgaacttcagggt

SEQ ID NO: 11 - RNA, 27 nt, Artificial Sequence
  synthetic
cgguggugcagaugaacuucaggguca

SEQ ID NO: 12 - DNA, 41 bp, Artificial Sequence
  synthetic
tgaccctgaagttcatctgcaccaccgcgagttgtaatggc

SEQ ID NO: 13 - RNA, 25 nt, Artificial Sequence
  synthetic
acccugaaguucaucugcaccaccg

SEQ ID NO: 14 - DNA, 39 bp, Artificial Sequence
  synthetic
ttgtctccgggacgcggtggtgcagatgaacttcagggt

SEQ ID NO: 15 - RNA, 27 nt, Artificial Sequence
  synthetic
cgguggugcagaugaacuucaggguca

SEQ ID NO: 16 - DNA, 42 bp, Artificial Sequence
  synthetic
tgaccctgaagttcatctgcaccaccggcgagttgtaatggc

SEQ ID NO: 17 - RNA, 25 nt, Artificial Sequence
  synthetic
acccugaaguucaucugcaccaccg

SEQ ID NO: 18 - DNA, 40 bp, Artificial Sequence
  synthetic
ttgtctccgggacgccggtggtgcagatgaacttcagggt

SEQ ID NO: 19 - RNA, 27 nt, Artificial Sequence
  synthetic
cgguggugcagaugaacuucaggguca

SEQ ID NO: 20 - DNA, 43 bp, Artificial Sequence
  synthetic
tgaccctgaagttcatctgcaccaccgggcgagttgtaatggc

SEQ ID NO: 21 - RNA, 25 nt, Artificial Sequence
  synthetic
acccugaaguucaucugcaccaccg

SEQ ID NO: 22 - DNA, 41 bp, Artificial Sequence
  synthetic
ttgtctccgggacgcccggtggtgcagatgaacttcagggt

SEQ ID NO: 23 - RNA, 27 nt, Artificial Sequence
  synthetic
cgguggugcagaugaacuucaggguca

SEQ ID NO: 24 - DNA, 39 bp, Artificial Sequence
  synthetic
tgaccctgaagttcatctgcaccaccgaagatgtcattg

SEQ ID NO: 25 - RNA, 27 nt, Artificial Sequence
  synthetic
cgguggugcagaugaacuucaggguca

SEQ ID NO: 26 - DNA, 43 bp, Artificial Sequence
  synthetic
tgaccctgaagttcatctgcaccaccgaagatgtcattgtctc

SEQ ID NO: 27 - RNA, 25 nt, Artificial Sequence
  synthetic
acccugaaguucaucugcaccaccg

SEQ ID NO: 28 - DNA, 79 bp, Artificial Sequence
  synthetic
tcctgtagtacagcgattcaaagatgtcattgtctcaacccaatgacatcttcggtggtgcagatgaacttcagggtca

SEQ ID NO: 29 - RNA, 25 nt, Artificial Sequence
  synthetic
acccugaaguucaucugcaccaccg

SEQ ID NO: 30 - DNA, 83 bp, Artificial Sequence
  synthetic
tcctgtagtacagcgattcaaagatgtcattgtctcaacaccatcaatgacatcttcggtggtgcagatgaacttcagggtca

SEQ ID NO: 31 - RNA, 25 nt, Artificial Sequence
  synthetic
acccugaaguucaucugcaccaccg

SEQ ID NO: 32 - DNA, 87 bp, Artificial Sequence
  synthetic
tcctgtagtacagcgattcaaagatgtcattgtctcaagcggacgagacaatgacatcttcggtggtgcagatgaacttcagggtca

SEQ ID NO: 33 - RNA, 25 nt, Artificial Sequence
  synthetic
acccugaaguucaucugcaccaccg

SEQ ID NO: 34 - DNA, 90 bp, Artificial Sequence
  synthetic
tagtacagcgattcaaagatgtcattgtctccgggaagcggaccccggagacaatgacatcttcggtggtgcagatgaacttcagggtca

SEQ ID NO: 35 - RNA, 25 nt, Artificial Sequence
  synthetic
acccugaaguucaucugcaccaccg

SEQ ID NO: 36 - DNA, 35 bp, Artificial Sequence
  synthetic
tagtacagcgattcaaagatgtcattgtctccggg

SEQ ID NO: 37 - DNA, 47 bp, Artificial Sequence
  synthetic
cccggagacaatgacatcttcggtggtgcagatgaacttcagggtca

SEQ ID NO: 38 - RNA, 27 nt, Artificial Sequence
  synthetic
cgguggugcagaugaacuucaggguca

SEQ ID NO: 39 - DNA, 90 bp, Artificial Sequence
  synthetic
tgaccctgaagttcatctgcaccaccgaagatgtcattggcaatgagggaccacaatgacatctttggtccctcattgcactgtactcct

SEQ ID NO: 40 - RNA, 27 nt, Artificial Sequence
  synthetic
cgguggugcagaugaacuucaggguca

SEQ ID NO: 41 - DNA, 39 bp, Artificial Sequence
  synthetic
tgaccctgaagttcatctgcaccaccgactgtaatgcta

SEQ ID NO: 42 - RNA, 25 nt, Artificial Sequence
  synthetic
acccugaaguucaucugcaccaccg

SEQ ID NO: 43 - DNA, 37 bp, Artificial Sequence
  synthetic
caatgacatcttcggtggtgcagatgaacttcagggt

SEQ ID NO: 44 - RNA, 25 nt, Artificial Sequence
  synthetic
acccugaaguucaucugcaccaccg

SEQ ID NO: 45 - DNA, 90 bp, Artificial Sequence
  synthetic
agatgtcattgtctccgggacagttgtactgtaatgctaacaactgtcccggatagcattacagtcggtggtgcagatgaacttcagggt

SEQ ID NO: 46 - RNA, 27 nt, Artificial Sequence
  synthetic
cgguggugcagaugaacuucaggguca

SEQ ID NO: 47 - DNA, 65 bp, Artificial Sequence
  synthetic
tgaccctgaagttcatctgcaccaccgaagatgtcattggcaatgagggaccacaatgacatctt

SEQ ID NO: 48 - DNA, 25 bp, Artificial Sequence
  synthetic
tggtccctcattgcactgtactcct

SEQ ID NO: 49 - RNA, 27 nt, Artificial Sequence
  synthetic
cgguggugcagaugaacuucaggguca

SEQ ID NO: 50 - DNA, 69 bp, Artificial Sequence
  synthetic
tgaccctgaagttcatctgcaccaccgaagatgtcattggcaatgagggaccacaatgacatctttggt

SEQ ID NO: 51 - DNA, 21 bp, Artificial Sequence
  synthetic
ccctcattgcactgtactcct

SEQ ID NO: 52 - RNA, 27 nt, Artificial Sequence
  synthetic
cgguggugcagaugaacuucaggguca

SEQ ID NO: 53 - DNA, 71 bp, Artificial Sequence
  synthetic
tgaccctgaagttcatctgcaccaccgaagatgtcattggcaatgagggaccacaatgacatctttggtcc

SEQ ID NO: 54 - DNA, 19 bp, Artificial Sequence
  synthetic
ctcattgcactgtactcct

SEQ ID NO: 55 - RNA, 27 nt, Artificial Sequence
  synthetic
cgguggugcagaugaacuucaggguca

SEQ ID NO: 56 - DNA, 73 bp, Artificial Sequence
  synthetic
tgaccctgaagttcatctgcaccaccgaagatgtcattggcaatgagggaccacaatgacatctttggtccct

SEQ ID NO: 57 - DNA, 17 bp, Artificial Sequence
  synthetic
cattgcactgtactcct

SEQ ID NO: 58 - DNA, 4 bp, Artificial Sequence
  synthetic
ggga

SEQ ID NO: 59 - RNA, 77 nt, Artificial Sequence
  synthetic
aagaccugugccugccauuacaacugucccggagacaaugacaucuuugaaucgcuguacuacaggaagauguacgg

SEQ ID NO: 60 - DNA, 3 bp, Artificial Sequence
  synthetic
ggg

SEQ ID NO: 61 - RNA, 60 nt, Artificial Sequence
  synthetic
cucgacacagcaggucaagaggaguacagugcaaugagggaccaguacaugaggacuggg

SEQ ID NO: 62 - RNA, 81 nt, Artificial Sequence
  synthetic
gggaaagaccugugccugccauuacaacugucccggagacaaugacaucuuugaaucgcuguacuacaggaagauguacgg